SEQUENCE LISTING (56 sequences)

SEQ ID NO: 1 - protein, 157 aa, synthetic construct [sequence omitted]

SEQ ID NO: 2 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, synthetic construct
GSIFSINDMG

SEQ ID NO: 4 - protein, 10 aa, synthetic construct
AIGRGGGSTN

SEQ ID NO: 5 - protein, 12 aa, synthetic construct
EISQLTWAFRDY

SEQ ID NO: 6 - protein, 121 aa, synthetic construct
EVQLVESGGGLVQPGGSLRLSCAASGSIFSINDMGWYRQAPGKQRELVAAIGRGGGSTNYADSVKGRFTISRDNAKNTVYLQMNSLKPEDTAVYYCHAEISQLTWAFRDYWGQGTQVTVSS

SEQ ID NO: 7 - protein, 121 aa, synthetic construct
EVQLVESGGGLVQPGGSLRLSCAASGSIFSINDMGWYRQAPGKGLELVAAIGRGGGSTNYADSVKGRFTISRDNAKNTLYLQMNSLRAEDTAVYYCHAEISQLTWAFRDYWGQGTLVTVSS

SEQ ID NO: 8 - protein, 121 aa, synthetic construct
EVQLVESGGGLVQPGGSLRLSCAASGSIFSINDMGWYRQAPGKQRELVAAIGRGGGSTNYADSVKGRFTISRDNAKNTLYLQMNSLRAEDTAVYYCHAEISQLTWAFRDYWGQGTLVTVSS

SEQ ID NO: 9 - protein, 461 aa, synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 10 - protein, 10 aa, synthetic construct
GSIFSINDMG

SEQ ID NO: 11 - protein, 10 aa, synthetic construct
VHGRGGGSTN

SEQ ID NO: 12 - protein, 12 aa, synthetic construct
EISQLTWAFRDY

SEQ ID NO: 13 - protein, 121 aa, synthetic construct
EVQLVESGGGLVQPGGSLRLSCAASGSIFSINDMGWYRQAPGKGLELVAVHGRGGGSTNYADSVKGRFTISRDNAKNTLYLQMNSLRAEDTAVYYCHAEISQLTWAFRDYWGQGTLVTVSS

SEQ ID NO: 14 - protein, 10 aa, synthetic construct
GSIFSINDMG

SEQ ID NO: 15 - protein, 10 aa, synthetic construct
AIGRGRRSTN

SEQ ID NO: 16 - protein, 12 aa, synthetic construct
EISQLSFAFRDY

SEQ ID NO: 17 - protein, 121 aa, synthetic construct
EVQLVESGGGLVQPGGSLRLSCAASGSIFSINDMGWYRQAPGKGLELVAAIGRGRRSTNYADSVKGRFTISRDNAKNTLYLQMNSLRAEDTAVYYCHAEISQLSFAFRDYWGQGTLVTVSS

SEQ ID NO: 18 - protein, 10 aa, synthetic construct
GSIFSINDMG

SEQ ID NO: 19 - protein, 10 aa, synthetic construct
AIGRGGQRTN

SEQ ID NO: 20 - protein, 12 aa, synthetic construct
EISQLSFAFRDY

SEQ ID NO: 21 - protein, 121 aa, synthetic construct
EVQLVESGGGLVQPGGSLRLSCAASGSIFSINDMGWYRQAPGKGLELVAAIGRGGQRTNYADSVKGRFTISRDNAKNTLYLQMNSLRAEDTAVYYCHAEISQLSFAFRDYWGQGTLVTVSS

SEQ ID NO: 22 - protein, 10 aa, synthetic construct
GSIFSILRMG

SEQ ID NO: 23 - protein, 10 aa, synthetic construct
AIGRTRGSTN

SEQ ID NO: 24 - protein, 12 aa, synthetic construct
EISQLTWAFRDY

SEQ ID NO: 25 - protein, 121 aa, synthetic construct
EVQLVESGGGLVQPGGSLRLSCAASGSIFSILRMGWYRQAPGKGLELVAAIGRTRGSTNYADSVKGRFTISRDNAKNTLYLQMNSLRAEDTAVYYCHAEISQLTWAFRDYWGQGTLVTVSS

SEQ ID NO: 26 - protein, 10 aa, synthetic construct
GSIWSINDMG

SEQ ID NO: 27 - protein, 10 aa, synthetic construct
AIGRGGGSTN

SEQ ID NO: 28 - protein, 12 aa, synthetic construct
EISQLTWAFLDY

SEQ ID NO: 29 - protein, 121 aa, synthetic construct
EVQLVESGGGLVQPGGSLRLSCAASGSIWSINDMGWYRQAPGKGLELVAAIGRGGGSTNYADSVKGRFTISRDNAKNTLYLQMNSLRAEDTAVYYCHAEISQLTWAFLDYWGQGTLVTVSS

SEQ ID NO: 30 - protein, 10 aa, synthetic construct
GSIFSINDMG

SEQ ID NO: 31 - protein, 10 aa, synthetic construct
AIGRRPGSTN

SEQ ID NO: 32 - protein, 12 aa, synthetic construct
EISQLSFAFRDY

SEQ ID NO: 33 - protein, 121 aa, synthetic construct
EVQLVESGGGLVQPGGSLRLSCAASGSIFSINDMGWYRQAPGKGLELVAAIGRRPGSTNYADSVKGRFTISRDNAKNTLYLQMNSLRAEDTAVYYCHAEISQLSFAFRDYWGQGTLVTVSS

SEQ ID NO: 34 - protein, 10 aa, synthetic construct
GSIFSILSMG

SEQ ID NO: 35 - protein, 10 aa, synthetic construct
AIGRGGGSLQ

SEQ ID NO: 36 - protein, 12 aa, synthetic construct
EISQLTWAFRDY

SEQ ID NO: 37 - protein, 121 aa, synthetic construct
EVQLVESGGGLVQPGGSLRLSCAASGSIFSILSMGWYRQAPGKGLELVAAIGRGGGSLQYADSVKGRFTISRDNAKNTLYLQMNSLRAEDTAVYYCHAEISQLTWAFRDYWGQGTLVTVSS

SEQ ID NO: 38 - protein, 10 aa, synthetic construct
GSIFSINDMG

SEQ ID NO: 39 - protein, 10 aa, synthetic construct
AIGRGSVSTN

SEQ ID NO: 40 - protein, 12 aa, synthetic construct
EISQLTYAFRDY

SEQ ID NO: 41 - protein, 121 aa, synthetic construct
EVQLVESGGGLVQPGGSLRLSCAASGSIFSINDMGWYRQAPGKGLELVAAIGRGSVSTNYADSVKGRFTISRDNAKNTLYLQMNSLRAEDTAVYYCHAEISQLTYAFRDYWGQGTLVTVSS

SEQ ID NO: 42 - protein, 10 aa, synthetic construct
GSIFSINDMG

SEQ ID NO: 43 - protein, 10 aa, synthetic construct
AIGRGGFSTN

SEQ ID NO: 44 - protein, 12 aa, synthetic construct
EISQLSFAFRDY

SEQ ID NO: 45 - protein, 121 aa, synthetic construct
EVQLVESGGGLVQPGGSLRLSCAASGSIFSINDMGWYRQAPGKGLELVAAIGRGGFSTNYADSVKGRFTISRDNAKNTLYLQMNSLRAEDTAVYYCHAEISQLSFAFRDYWGQGTLVTVSS

SEQ ID NO: 46 - protein, 10 aa, synthetic construct
GSIRFINDMG

SEQ ID NO: 47 - protein, 10 aa, synthetic construct
AIGRGGGSTN

SEQ ID NO: 48 - protein, 12 aa, synthetic construct
EISQLGYAFRDY

SEQ ID NO: 49 - protein, 121 aa, synthetic construct
EVQLVESGGGLVQPGGSLRLSCAASGSIRFINDMGWYRQAPGKGLELVAAIGRGGGSTNYADSVKGRFTISRDNAKNTLYLQMNSLRAEDTAVYYCHAEISQLGYAFRDYWGQGTLVTVSS

SEQ ID NO: 50 - protein, 10 aa, synthetic construct
GSIFSINDMG

SEQ ID NO: 51 - protein, 10 aa, synthetic construct
ALARGGGSTN

SEQ ID NO: 52 - protein, 12 aa, synthetic construct
EISQLSFAFRDY

SEQ ID NO: 53 - protein, 121 aa, synthetic construct
EVQLVESGGGLVQPGGSLRLSCAASGSIFSINDMGWYRQAPGKGLELVAALARGGGSTNYADSVKGRFTISRDNAKNTLYLQMNSLRAEDTAVYYCHAEISQLSFAFRDYWGQGTLVTVSS

SEQ ID NO: 54 - protein, 10 aa, synthetic construct
GSIXXIXXMG

SEQ ID NO: 55 - skipped

SEQ ID NO: 56 - protein, 12 aa, synthetic construct
EISQLXXAFXDY

(19) United States
(12) Patent Application Publication
(10) Pub. No.: US 2025/0043016 A1
(43) Pub. Date: Feb. 6, 2025

(54) TNFR2 BINDING MOLECULE AND USE THEREOF

(71) Applicant: SANYOU BIOPHARMACEUTICALS CO., LTD., Shanghai (CN)

(72) Inventors: Guojun LANG, Shanghai (CN); Chao KONG, Shanghai (CN); Yuhao HU, Shanghai (CN); Xiaoxiao SHI, Shanghai (CN); Zhen ZHANG, Shanghai (CN); Xintian YAN, Shanghai (CN); Run YAN, Shanghai (CN); Chanjuan LIU, Shanghai (CN); Yongcong TAN, Shanghai (CN)

(21) Appl. No.: 18/716,868

(22) PCT Filed: Dec. 5, 2022
(86) PCT No.: PCT/CN2022/136599

§ 371 Date: Jun. 5, 2024

(30) Foreign Application Priority Data
Dec. 6, 2021 (CN) 202111477145.7

(51) Int. Cl. C07K 16/28; A61K 39/00; A61P 35/00; G01N 33/68

(52) U.S. Cl. CPC C07K 16/2878; A61P 35/00; C07K 16/2818; G01N 33/6854; A61K 2039/505; C07K 2317/22; C07K 2317/24; C07K 2317/31; C07K 2317/33; C07K 2317/565; C07K 2317/569; C07K 2317/76; G01N 2333/7151

(57) ABSTRACT

The present invention relates to a specific TNFR2 binding molecule, an epitope peptide of TNFR2 that is bound to the TNFR2 binding molecule, and a composition containing same. The present invention also relates to a nucleic acid encoding the TNFR2 binding molecule, a host cell containing the nucleic acid, and a method for preparing the TNFR2 binding molecule. Furthermore, the present invention relates to the therapeutic and diagnostic use of the TNFR2 binding molecules. Particularly, the present invention relates to the combined treatment of the TNFR2 binding molecules with other therapies, such as therapeutic methods or therapeutic agents.

### TECHNICAL FIELD

The present invention relates to a specific TNFR2 binding molecule, an epitope peptide of TNFR2 that is bound by the TNFR2 binding molecule, and a composition containing the TNFR2 binding molecule. Furthermore, the present invention relates to a nucleic acid encoding the TNFR2 binding molecule, a host cell containing the nucleic acid, and a method for preparing the TNFR2 binding molecule. The present invention further relates to the therapeutic and diagnostic use of the TNFR2 binding molecules. Particularly, the present invention further relates to the combined treatment of the TNFR2 binding molecules with other therapies, such as therapeutic methods or therapeutic agents.

### BACKGROUND ART

Tumor necrosis factor receptor 2 (TNFR2, TNFRSF1B), belonging to the tumor necrosis factor receptor superfamily, is expressed on the surfaces of activated regulatory T cells (Tregs), myeloid-derived suppressing cells (MDSCs), and CD4- and CD8-positive effector T cells, and is also highly expressed on the surfaces of various tumor cells, such as Sézary syndrome and granuloma fungoides (Medler J., Wajant H. (2019). Expert Opin Ther Targets 23, 295-307.). Unlike the widespread expression of TNFR1, TNFR2 is generally more specifically expressed, particularly highly upregulated in tumor infiltrating immune cells such as regulatory T cells (Tregs), cytotoxic T cells, and different subsets of myeloid cells (Sheng Y., Li F., Qin Z. (2018). Front Immunol 9, 1170.). TNFR2 positive Treg cells are highly enriched in many tumors, causing a highly suppressive immune microenvironment local to the tumor tissue, while the TNFR2 positive Tregs also exhibit active immunosuppressive activity, becoming a major obstacle to anti-tumor immune responses in the tumor microenvironment (Yang Y., Islam M. S., Hu Y., Chen X. (2021). Immunotargets Ther 10, 103-122.). Since TNFR2 is specifically highly expressed on the surfaces of intratumoral Tregs, MDSCs and many tumor cells, it is expected to be a promising target for cancer immunotherapy, bringing better efficacy and higher safety.

Studies have shown that an antagonistic antibody drug targeting TNFR2 can activate anti-tumor immune responses by inhibiting or killing intratumoral immunosuppressive cells such as Tregs and MDSCs, achieving the therapeutic effect of killing tumors (Sheng Y., Li F., Qin Z. (2018). Front Immunol 9, 1170.).

Although there are antagonistic monoclonal antibody drugs (e.g., BI-1808) targeting TNFR2 under current clinical research, there are problems of poor therapeutic effect and high toxicity. There is still an urgent need to continue to develop small molecule antibodies (e.g., single domain antibodies) targeting TNFR2 as therapeutic agents.

### SUMMARY OF THE INVENTION

The present invention develops a class of TNFR2 binding molecules containing a single domain antibody (sdAb) moiety that specifically recognizes TNFR2, having one or more of the following properties:
(1) binding to human TNFR2 with high affinity, for example, the EC₅₀ for the binding between the TNFR2 binding molecule and cell surface TNFR2 is about 0.01 μg/mL to about 1 μg/mL, such as about 0.1 μg/ml to about 0.6 μg/ml;
(2) not substantially blocking the binding of TNFα to TNFR2;
(3) inhibiting the TNFR2 signaling pathway, for example, signaling mediated by TNFR2 is inhibited in TNFR2-expressing cells such as Treg cells (e.g., Treg cells with high expression of CD25), myeloid-derived suppressing cells (MDSCs) and/or TNFR2+ cancer cells;
(4) not substantially affecting the proliferation of Treg cells in PBMCs;
(5) inhibiting tumor growth in vivo.

Therefore, in a first aspect, the present invention provides a TNFR2 binding molecule, containing at least one single domain antibody (sdAb) moiety that specifically binds to TNFR2, wherein the sdAb moiety contains, from the N-terminus to the C-terminus, three complementarity determining regions, respectively CDR1, CDR2 and CDR3, wherein:
(a) CDR1 contains an amino acid sequence of SEQ ID NO: 3, or a variant with 1 or 2 amino acid changes in the amino acid sequence of SEQ ID NO: 3,
(b) CDR2 contains an amino acid sequence of SEQ ID NO: 4, or a variant with 1 or 2 amino acid changes in the amino acid sequence of SEQ ID NO: 4, and
(c) CDR3 contains an amino acid sequence of SEQ ID NO: 5, or a variant with 1 or 2 amino acid changes in the amino acid sequence of SEQ ID NO: 5,
wherein the amino acid change is an addition, deletion or substitution of an amino acid, and the binding molecule containing the above-mentioned change at least retains the binding ability to TNFR2.

In some embodiments, the sdAb moiety of the TNFR2 binding molecule of the present invention contains
(a) CDR1 containing an amino acid sequence of SEQ ID NO: 54:

(SEQ ID NO: 54)
G-S-I-Xaa1-Xaa2-I-Xaa3-Xaa4-M-G

wherein Xaa1 is F, W or R, Xaa2 is S or F, Xaa3 is N or L, and Xaa4 is S, D or R;
(b) CDR2 containing an amino acid sequence of SEQ ID NO: 55:

(SEQ ID NO: 55)
Xaa5-Xaa6-Xaa7-R-Xaa8-Xaa9-Xaa10-Xaa11-Xaa12-
Xaa13

wherein Xaa5 is A or V, Xaa6 is I, L or H, Xaa7 is G or A, Xaa8 is G, R or T, Xaa9 is G, R, P or S, Xaa10 is G, Q, R, For V, Xaa11 is S or R, Xaa12 is T or L, and Xaa13 is N or Q; and
(c) CDR3 containing an amino acid sequence of SEQ ID NO: 56:

(SEQ ID NO: 56)
E-I-S-Q-L-Xaa14-Xaa15-A-F-Xaa16-D-Y

wherein Xaa14 is T, S or G, Xaa15 is W, F or Y, and Xaa16 is R or L.

In some embodiments, the sdAb moiety of the TNFR2 binding molecule of the present invention contains CDR1, CDR2 and CDR3 selected from any one of the following groups:
(a) CDR1 containing an amino acid sequence of SEQ ID NO: 3; CDR2 containing an amino acid sequence of SEQ ID NO: 4; and CDR3 containing an amino acid sequence of SEQ ID NO: 5;
(b) CDR1 containing an amino acid sequence of SEQ ID NO: 10; CDR2 containing an amino acid sequence of SEQ ID NO: 11; and CDR3 containing an amino acid sequence of SEQ ID NO: 12;
(c) CDR1 containing an amino acid sequence of SEQ ID NO: 14; CDR2 containing an amino acid sequence of SEQ ID NO: 15; and CDR3 containing an amino acid sequence of SEQ ID NO: 16;
(d) CDR1 containing an amino acid sequence of SEQ ID NO: 18; CDR2 containing an amino acid sequence of SEQ ID NO: 19; and CDR3 containing an amino acid sequence of SEQ ID NO: 20;
(e) CDR1 containing an amino acid sequence of SEQ ID NO: 22; CDR2 containing an amino acid sequence of SEQ ID NO: 23; and CDR3 containing an amino acid sequence of SEQ ID NO: 24;
(f) CDR1 containing an amino acid sequence of SEQ ID NO: 26; CDR2 containing an amino acid sequence of SEQ ID NO: 27; and CDR3 containing an amino acid sequence of SEQ ID NO: 28;
(g) CDR1 containing an amino acid sequence of SEQ ID NO: 30; CDR2 containing an amino acid sequence of SEQ ID NO: 31; and CDR3 containing an amino acid sequence of SEQ ID NO: 32;
(h) CDR1 containing an amino acid sequence of SEQ ID NO: 34; CDR2 containing an amino acid sequence of SEQ ID NO: 35; and CDR3 containing an amino acid sequence of SEQ ID NO: 36;
(i) CDR1 containing an amino acid sequence of SEQ ID NO: 38; CDR2 containing an amino acid sequence of SEQ ID NO: 39; and CDR3 containing an amino acid sequence of SEQ ID NO: 40;
(j) CDR1 containing an amino acid sequence of SEQ ID NO: 42; CDR2 containing an amino acid sequence of SEQ ID NO: 43; and CDR3 containing an amino acid sequence of SEQ ID NO: 44;
(k) CDR1 containing an amino acid sequence of SEQ ID NO: 46; CDR2 containing an amino acid sequence of SEQ ID NO: 47; and CDR3 containing an amino acid sequence of SEQ ID NO: 48;
(l) CDR1 containing an amino acid sequence of SEQ ID NO: 50; CDR2 containing an amino acid sequence of SEQ ID NO: 51; and CDR3 containing an amino acid sequence of SEQ ID NO: 52.

In some embodiments, the sdAb moiety of the TNFR2 binding molecule of the present invention contains
(i) an amino acid sequence selected from any one of amino acid sequences of SEQ ID NOs: 6, 7, 8, 13, 17, 21, 25, 29, 33, 37, 41, 45, 49 and 53; or
(ii) an amino acid sequence having at least 85%, 90%, 91%, 92%, 93%, 94%, 95%, 96%, 97%, 98% or 99% identity to the amino acid sequence selected from any one of amino acid sequences of SEQ ID NOs: 6, 7, 8, 13, 17, 21, 25, 29, 33, 37, 41, 45, 49 and 53;
preferably, the sdAb moiety is camelid VHH, partially humanized or fully humanized VHH, or chimeric VHH.

In some embodiments, the TNFR2 binding molecule of the present invention is further linked to another protein domain at the N-terminus or C-terminus of the sdAb moiety, for example, to the Fc region of an immunoglobulin, for example, to the Fc region from IgG, such as IgG1, IgG2, IgG3 or IgG 4; or, for example, the sdAb moiety is linked to a fluorescent protein.

In some embodiments, the TNFR2 binding molecule of the present invention is a bispecific or multispecific antibody, preferably, the bispecific antibody molecule specifically binds to a TNFR2 molecule and a second target protein, and the second target protein is selected from, for example, a tumor antigen (such as a tumor associated antigen and a tumor specific antigen), an immunoregulatory receptor, and an immune checkpoint molecule, such as CTLA-4, TIM-3 or LAG-3.

In a second aspect, the present invention provides a method for preparing the TNFR2 binding molecule of the present invention, wherein the method comprises culturing a host cell introduced with a nucleic acid encoding the TNFR2 binding molecule of the present invention or an expression vector containing the nucleic acid under conditions suitable for expressing a nucleic acid encoding the TNFR2 binding molecule of the present invention, and isolating the TNFR2 binding molecule, and optionally the method further comprises recovering the TNFR2 binding molecule from the host cell.

In a third aspect, the present invention provides a pharmaceutical composition, containing the TNFR2 binding molecule of the present invention, and optionally a pharmaceutical auxiliary material.

In some embodiments, the present invention provides a pharmaceutical composition, containing the TNFR2 binding molecule of the present invention, and other therapeutic agents, and optionally a pharmaceutical auxiliary material, wherein preferably, the other therapeutic agents are selected from chemotherapeutic agents and other antibodies (such as anti-PD-1 antibodies or anti-PD-L1 antibodies).

In some embodiments, the present invention provides a combined product, containing the TNFR2 binding molecule of the present invention, and one or more other therapeutic agents, such as chemotherapeutic agents and other antibodies, such as anti-PD-1 antibodies or anti-PD-L1 antibodies.

In a fourth aspect, the present invention provides a method for treating a disease associated with TNFR2 in a subject, comprising administering to the subject a therapeutically effective amount of the TNFR2 binding molecule, the pharmaceutical composition, or the combined product of the present invention.

In some embodiments, the disease associated with high expression of TNFR2 treated by the TNFR2 binding molecule, the pharmaceutical composition, or the combined product of the present invention is, for example, a cancer that expresses or overexpresses TNFR2.

In a fifth aspect, the present invention provides a kit for detecting TNFR2 in a sample, wherein the kit contains the TNFR2 binding molecule of the present invention, and is used for performing the following steps:
(a) contacting the sample with the TNFR2 binding molecule of the present invention; and
(b) detecting the formation of a complex of the TNFR2 binding molecule and TNFR2; optionally, the TNFR2 binding molecule is detectably labeled,
and therefore, it is determined whether an elevated TNFR2 expression level is present in the sample from the subject or individual.

In a sixth aspect, the present invention provides an epitope peptide of TNFR2 that is bound by the TNFR2 binding molecule of the present invention, wherein the epitope peptide is located in the groove of the CRD3 domain of TNFR2, for example, the epitope peptide is an epitope peptide of TNFR2 that contains amino acid residues at positions 83, 84, 85, 97, 98, 100, 101, 108, 110, 112, 131, 132 and 133, for example, the epitope peptide is an epitope peptide of TNFR2 as shown in SEQ ID NO: 9 that contains amino acid residues at positions V83, E84, T85, T97, C98, P100, G101, K108, E110, C112, G131, T132 and E133.

In a seventh aspect, the present invention provides a TNFR2 binding molecule, wherein the TNFR2 binding molecule binds in the groove of the CRD3 domain of TNFR2, for example, the TNFR2 binding molecule binds to an epitope of TNFR2 that contains amino acid residues at positions 83, 84, 85, 97, 98, 100, 101, 108, 110, 112, 131, 132 and 133, for example, the TNFR2 binding molecule binds to an epitope of TNFR2 as shown in SEQ ID NO: 9 that contains amino acid residues at positions V83, E84, T85, T97, C98, P100, G101, K108, E110, C112, G131, T132 and E133.

### BRIEF DESCRIPTION OF THE DRAWINGS

The following detailed description of the preferred embodiments of the present invention can be better understood when reading in conjunction with the following drawings below. For the purpose of illustrating the present invention, there are shown in the drawings embodiments which are presently preferred. However, it should be understood that the present invention is not limited to the precise arrangements and instrumentalities of the embodiments shown in the drawings.

FIG. 1 shows the FACS identification result of huTNFR2-HEK293 cell line.

FIG. 2 shows the FACS identification result of huTNFR2-Jurkat cell line.

FIG. 3 shows the binding of candidate positive clone lysate to recombinant human TNFR2.

FIG. 4 shows the binding activity of anti-TNFR2 VHH-Fc chimeric antibody to huTNFR2-HEK293 cells.

FIG. 5 shows the species cross-reactivity of anti-TNFR2 VHH-Fc chimeric antibody.

FIG. 6 shows the blocking activity of anti-TNFR2 VHH-Fc chimeric antibody against the binding of TNFα to huTNFR2-HEK293 cells (the chimeric antibody does not substantially block the binding).

FIG. 7 shows the inhibitory activity of anti-TNFR2 VHH-Fc chimeric antibody on TNFα-induced huTNFR2-Jurkat cell necrosis.

FIG. 8 shows the inhibitory effect of anti-TNFR2 VHH-Fc chimeric antibody on tumor growth in humanized mice.

FIG. 9 shows the binding activity of anti-TNFR2 humanized antibody to huTNFR2-HEK293 cells.

FIGS. 10A-10D show the binding activities of anti-TNFR2 affinity-matured molecules to huTNFR2-HEK293 cells.

FIG. 11 shows the blocking activity of anti-TNFR2 affinity-matured molecule against the binding of TNFα to huTNFR2-HEK293 cells (the affinity-matured molecule does not substantially block the binding).

FIG. 12 shows the inhibitory activity of anti-TNFR2 affinity-matured molecule on TNFα-induced huTNFR2-Jurkat cell necrosis.

FIG. 13 shows the effect of anti-TNFR2 affinity-matured molecule on the proliferation of Treg cells in PBMCs (the affinity-matured molecule does not affect the proliferation of Treg cells in normal PBMCs).

FIG. 14 shows the inhibitory effect of anti-TNFR2 affinity-matured molecule on tumor growth in humanized mice.

FIG. 15A shows the ADCC effect of affinity-matured molecule 161-hVH5-48 on huTNFR2-HEK293 cells compared with control antibody.

FIG. 15B shows the ADCC effect of affinity-matured molecule 161-hVH5-48 on huTNFR2-Jurkat cells compared with control antibody.

FIG. 16 shows the X-ray diffraction analysis result of complex crystal produced by complexing 161-hVH5-48 antibody with TNFR2.

### DETAILED DESCRIPTION OF EMBODIMENTS

Unless otherwise defined, all technical and scientific terms used herein have the same meanings as commonly understood by those of ordinary skill in the art to which the present invention pertains. All publications, patent applications, patents and other references mentioned herein are incorporated by reference in its entirety. In addition, the materials, methods and examples described herein are illustrative only and are not intended to be limiting. Other features, objectives and advantages of the present invention will be apparent from the description, the drawings and the appended claims.

### I. Definitions

For interpreting this description, the following definitions will apply and whenever appropriate, terms used in the singular will also include the plural and vice versa. It is to be understood that the terminology used herein is for the purpose of describing particular embodiments only, and is not intended to be limiting.

Unless otherwise indicated, the term “contain” or “comprise” when used herein also encompasses situations consisting of the described elements, integers or steps. For example, when referring to an antibody variable region “containing” a particular sequence, it is intended to also encompass an antibody variable region consisting of that particular sequence.

As used herein, the terms “TNFR2 antibody”, “anti-TNFR2 antibody”, “antibody that specifically binds to TNFR2”, “antibody that specifically targets TNFR2”, and “antibody that specifically recognizes TNFR2” are used interchangeably, and mean antagonistic TNFR2 antibodies that can specifically bind to TNFR2. Particularly, in a specific embodiment, the terms mean antagonistic TNFR2 antibodies that specifically bind to human TNFR2. The antagonistic TNFR2 antibody refers to a TNFR2 antibody that can inhibit or reduce the activation of TNFR2, attenuate one or more signaling pathways mediated by TNFR2, and/or reduce or inhibit at least one activity mediated by the activation of TNFR2. For example, the antagonistic TNFR2 antibody can inhibit or reduce the growth and proliferation of regulatory T cells.

The term “antibody” herein is used in the broadest sense, refers to a protein containing an antigen binding site, and encompasses natural antibodies and artificial antibodies of various structures, including but not limited to monoclonal antibodies, polyclonal antibodies, multispecific antibodies (such as bispecific antibodies), single chain antibodies, intact antibodies, and antibody fragments. Preferably, the antibody of the present invention is a single domain antibody, a chimeric antibody or a humanized antibody.

The term “antibody fragment” refers to a molecule different from an intact antibody, which contains a portion of the intact antibody and binds to an antigen that is bound by the intact antibody. Examples of the antibody fragments include, but are not limited to, Fv, Fab, Fab′, Fab′-SH, F(ab′)₂; diabodies; linear antibodies; single chain antibodies (such as scFv); single domain antibodies; bivalent or bispecific antibodies or fragments thereof; camelid antibodies (heavy chain antibodies); and bispecific or multispecific antibodies formed from antibody fragments.

The “complementarity determining region” or “CDR region” or “CDR” is a region of an antibody variable domain which is hypervariable in the sequence and forms a structurally defined loop (“hypervariable loop”) and/or contains an antigen contact residue (“antigen contact point”). CDRs are mainly responsible for binding to antigenic epitopes, and include, sequentially numbered, starting from the N-terminus, CDR1, CDR2 and CDR3. In a given variable region amino acid sequence, the precise amino acid sequence boundary of each CDR may be determined by using any one of many well-known antibody CDR assignment systems or a combination thereof, wherein the assignment system includes, for example: Chothia based on the three-dimensional structures of antibodies and the topology of CDR loops (Chothia et al., (1989) Nature 342:877-883, Al-Lazikani et al., “Standard conformations for the canonical structures of immunoglobulins”, Journal of Molecular Biology, 273, 927-948 (1997)), Kabat based on antibody sequence variability (Kabat et al., Sequences of Proteins of Immunological Interest, 4th edition, U.S. Department of Health and Human Services, National Institutes of Health (1987)), AbM (University of Bath), Contact (University College London), International ImMunoGeneTics database (IMGT) (http://imgt.cines.fr/), and the North CDR definition based on affinity propagation clustering using a large number of crystal structures. Unless otherwise stated, in the present invention, the term “CDR” or “CDR sequence” encompasses CDR sequences determined by any one of the above methods. CDR can also be determined on the basis of having the same AbM numbering position as a reference CDR sequence (such as any one sequence of the exemplary CDRs of the present invention). In one embodiment, the position of the CDR of the single domain antibody of the present invention is determined according to the AbM numbering scheme. Unless otherwise stated, in the present invention, when referring to residue positions (including heavy chain variable region residues) in antibody variable regions and CDRs, it is referred to as numbering positions according to the AbM numbering system.

Antibodies with different specificities (i.e., different binding sites for different antigens) have different CDRs. However, although CDRs vary from antibody to antibody, only a limited number of amino acid positions within the CDRs are directly involved in antigen binding. Using at least two of the Kabat, Chothia, IMGT, AbM and Contact methods, a minimal overlap region can be determined, thereby providing a “minimal binding unit” for antigen binding. The minimal binding unit may be a sub-portion of a CDR. As will be apparent to a person skilled in the art, the residues of the remainder of the CDR sequence can be determined from the structure and protein folding of an antibody. Therefore, the present invention also contemplates variants of any of the CDRs given herein. For example, in a variant of a CDR, the amino acid residue of the minimal binding unit can remain unchanged, while the remaining CDR residues defined according to Kabat or Chothia or AbM can be replaced with conserved amino acid residues.

The term “single domain antibody” generally refers to an antibody in which a single variable domain (such as a heavy chain variable domain (VH) or a light chain variable domain (VL), a heavy chain variable domain derived from a camelid heavy-chain antibody, and a VH-like single domain derived from fish IgNAR (v-NAR)) confers antigen binding. That is, the single variable domain does not need to interact with another variable domain to recognize a target antigen. Examples of the single domain antibody include those derived from camelid (llamas and camels) and cartilaginous fish (e.g., nurse sharks) (WO 2005035572A2). The single domain antibody derived from camelid, also referred to in the present application as VHH, consists of only one heavy chain variable region, is an antibody containing only one chain FR4-CDR3-FR3-CDR2-FR2-CDR1-FR1 from the C-terminus to the N-terminus, and is also called “nanobody”. The single domain antibody is the smallest unit currently known that can bind to a target antigen.

The “heavy-chain antibody (hcAb)” refers to an antibody without a light chain, which may contain VH-CH2-CH3, or contain VH-CH1-CH2-CH3, or contain VHH-CH2-CH3, from the N-terminus to the C-terminus; and a homodimer can be formed, such as a dimeric heavy-chain antibody without a light chain. The heavy-chain antibody may contain the VH from a standard antibody or the VHH from a single domain antibody. In one embodiment, the heavy-chain antibody of the present invention contains the VHH of a single domain antibody.

As used herein, the term “multispecific antibody” refers to an antibody having at least two antigen binding sites, each of which binds to a different epitope of the same antigen or a different epitope of a different antigen. The multispecific antibody is an antibody having binding specificities for at least two different antigenic epitopes. In one embodiment, provided herein is a bispecific antibody having binding specificities for a first antigen and a second antigen. As used herein, the “first antigen binding moiety” and “second antigen binding moiety” refer to an amino acid sequence that contains an antigen binding site and can bind to an antigenic epitope, the definition of which falls within the meaning of an antibody or an antigen binding fragment.

The term “chimeric antibody” is an antibody molecule in which (a) the constant region or a portion thereof is altered, replaced, or exchanged such that the antigen binding site is associated with a constant region belonging to different or changed classes, having different or changed effector functions and/or belonging to different or changed species, or a completely different molecule (such as enzymes, toxins, hormones, growth factors, and drugs) that gives a chimeric antibody new properties; or (b) the variable region or a portion thereof is altered, replaced, or exchanged with a variable region with a different or changed antigen specificity. For example, a camel antibody can be modified by replacing its constant region with a constant region from a human immunoglobulin. Due to the replacement with a human constant region, the chimeric antibody can retain its specificity in recognizing the antigen while having reduced antigenicity in humans as compared with the original camel antibody.

The “humanized antibody” refers to a chimeric antibody containing amino acid residues from non-human CDRs and amino acid residues from human FRs. In some embodiments, all or substantially all CDRs in a humanized antibody correspond to those in a non-human antibody, and all or substantially all FRs correspond to those in a human antibody. A humanized antibody may optionally contain at least a portion of an antibody constant region derived from a human antibody. A “humanized form” of an antibody (such as a non-human antibody) refers to an antibody that has undergone humanization.

The “human antibody” refers to an antibody having an amino acid sequence corresponding to that of an antibody produced by human or human cells or derived from a non-human source using human antibody repertoire or other human antibody coding sequences. This definition of the human antibody explicitly excludes a humanized antibody containing non-human antigen binding residues.

The term “Fc region” herein is used to define a C-terminal region of an immunoglobulin heavy chain, containing at least a portion of the constant region. The term includes natural sequence Fc regions and variant Fc regions. In certain embodiments, a human IgG heavy chain Fc region extends from Cys226 or Pro230 to the carbonyl terminus of the heavy chain. However, the C-terminal lysine (Lys447) of the Fc region may or may not be present. Unless otherwise stated, the amino acid residues in the Fc region or constant region are numbered according to the EU numbering system, also known as the EU index, as described in Kabat et al., Sequences of Proteins of Immunological Interest, 5th Ed. Public Health Service, National Institutes of Health, Bethesda, MD, 1991.

The term “variable region” or “variable domain” refers to an antibody heavy or light chain domain that is involved in the binding of an antibody to an antigen. The heavy and light chain variable domains of a natural antibody generally have similar structures, with each domain containing four conserved framework regions (FRs) and three complementarity determining regions (CDRs) (see, for example, Kindt et al., Kuby Immunology, 6^{th} ed., W.H.Freeman and Co., page 91 (2007)). A single VH or VL domain may be sufficient to confer an antigen binding specificity.

As used herein, the term “binding” or “specific binding” means that the binding is selective for an antigen and can be distinguished from unwanted or non-specific interactions. The ability of an antibody to bind to a particular antigen can be determined by enzyme-linked immunosorbent assay (ELISA), SPR or biolayer interferometry or other conventional binding assays known in the art.

The term “immune checkpoint molecule” refers to a class of inhibitory signaling molecules present in the immune system that avoid tissue damage by regulating the persistence and intensity of immune responses in peripheral tissues and participate in maintaining tolerance to self-antigens (Pardoll DM., The blockade of immune checkpoints in cancer immunotherapy. Nat Rev Cancer, 2012, 12 (4): 252-264). Research has found that one of the reasons why tumor cells are able to evade the immune system in the body and proliferate out of control is the use of inhibitory signaling pathways of immune checkpoint molecules, thereby inhibiting the activity of T lymphocytes and making T lymphocytes unable to effectively exert their killing effect on tumors (Yao S, Zhu Y and Chen L., Advances in targeting cell surface signaling molecules for immune modulation. Nat Rev Drug Discov, 2013, 12 (2): 130-146).

The term “therapeutically effective amount” refers to an amount effective to achieve the desired therapeutic result at a dose and for periods of time desired. The therapeutically effective amount of an antibody or an antibody fragment or a conjugate or a composition thereof can vary depending on a variety of factors such as disease state, age, sex and weight of an individual, and the ability of the antibody or antibody moiety to activate a desired response in an individual. The therapeutically effective amount is also an amount in which any toxic or harmful effect of an antibody or an antibody fragment or a conjugate or a composition thereof is less than a therapeutically beneficial effect. The “therapeutically effective amount” preferably inhibits measurable parameters (such as tumor growth rate and tumor volume) by at least about 20%, more preferably at least about 40%, even more preferably at least about 50%, 60% or 70%, and still more preferably at least about 80% or 90% relative to untreated subjects. The ability of a compound to inhibit a measurable parameter (such as cancer) can be evaluated in an animal model system for predicting the efficacy in a human tumor.

The terms “individual” and “subject” are used interchangeably, including mammals. Mammals include, but are not limited to, domesticated animals (e.g., cows, sheep, cats, dogs and horses), primates (e.g., humans and non-human primates such as monkeys), rabbits and rodents (e.g., mice and rats). Particularly, the individual or subject is a human.

The terms “tumor” and “cancer” are used interchangeably herein, and encompass solid tumors and liquid tumors.

The terms “cancer” and “cancerous” refer to the physiological illness in mammals in which cell growth is unregulated.

The term “tumor” refers to all neoplastic cell growth and proliferation, whether malignant or benign, and all pre-cancerous and cancerous cells and tissues. The terms “cancer”, “cancerous” and “tumor” are not mutually exclusive when referred to herein.

The “isolated nucleic acid” refers to a nucleic acid molecule that has been isolated from a component of the natural environment thereof. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. The “isolated nucleic acid encoding TNFR2 binding molecule” refers to one or more nucleic acid molecules encoding the chain or fragment of a TNFR2 binding molecule, including such nucleic acid molecules in a single vector or separate vectors, and such nucleic acid molecules present at one or more locations in a host cell.

The calculation of sequence identity between sequences is performed as follows.

To determine the percent identity of two amino acid sequences or two nucleic acid sequences, the sequences are aligned for optimal comparison purposes (for example, gaps can be introduced in either or both of the first and second amino acid sequences or nucleic acid sequences for optimal alignment or non-homologous sequences can be discarded for comparison purposes). In a preferred embodiment, the length of a reference sequence aligned for comparison purposes is at least 30%, preferably at least 40%, more preferably at least 50% or 60%, and even more preferably at least 70%, 80%, 90% or 100% of the length of the reference sequence. The amino acid residues or nucleotides at corresponding amino acid positions or nucleotide positions are then compared. When the position in the first sequence is occupied by the same amino acid residue or nucleotide as that at the corresponding position in the second sequence, the molecules are identical at that position.

The sequence comparison and the calculation of the percent identity between two sequences can be achieved using a mathematical algorithm. In a preferred embodiment, the percent identity between two amino acid sequences is determined using the Needlema and Wunsch algorithm ((1970) J. Mol. Biol. 48:444-453) in the GAP program that has been integrated into the GCG software package (available at http://www.gcg.com), and using the Blossum 62 matrix or the PAM250 matrix, with a gap weight of 16, 14, 12, 10, 8, 6 or 4 and a length weight of 1, 2, 3, 4, 5 or 6. In another preferred embodiment, the percent identity between two nucleotide sequences is determined using the GAP program in the GCG software package (available at http://www.gcg.com), and using the NWSgapdna.CMP matrix, with a gap weight of 40, 50, 60, 70 or 80 and a length weight of 1, 2, 3, 4, 5 or 6. A particularly preferred parameter set (and one parameter set that should be used unless otherwise stated) is the Blossum 62 scoring matrix with a gap penalty of 12, a gap extension penalty of 4 and a gap frameshift penalty of 5.

The percent identity between two amino acid sequences or nucleotide sequences can also be determined using the PAM120 weighted remainder table with a gap length penalty of 12 and a gap penalty of 4, and using the E. Meyers and W. Miller algorithm ((1989) CABIOS, 4:11-17) that has been incorporated into the ALIGN program (version 2.0).

Additionally or alternatively, the nucleic acid sequences and protein sequences described herein can further be used as “query sequences” to perform searches against public databases, for example, to identify other family member sequences or related sequences.

The term “transfection” refers to the process of introducing a nucleic acid into eukaryotic cells, particularly mammalian cells. Solutions and techniques for transfection include, but are not limited to, lipofection, and transfection using chemical and physical methods such as electroporation.

The term “disease associated with TNFR2” refers to any disorder induced by or exacerbated by or otherwise associated with increased expression or activity of TNFR2 (such as human TNFR2).

The term “pharmaceutical composition” refers to a composition that is present in a form which allows the active ingredient contained therein to be biologically effective and does not contain additional ingredients that would be unacceptably toxic to a subject to which the pharmaceutical composition is administered.

The term “pharmaceutical auxiliary material” refers to a diluent, an adjuvant (such as Freund's adjuvant (complete and incomplete)), a carrier, an excipient, a stabilizer, etc. administered with an active substance.

As used herein, the “treatment” refers to slowing, interrupting, blocking, relieving, stopping, reducing or reversing the progression or severity of an existing symptom, disorder, condition or disease. Desirable effects of treatment include, but are not limited to, preventing occurrence or recurrence of a disease, alleviating symptoms, diminishing any direct or indirect pathological consequences of a disease, preventing metastasis, decreasing the rate of disease progression, ameliorating or palliating disease state, and relieving or improving prognosis. In some embodiments, the antibody molecule of the present invention is used to delay the development of a disease or to slow the progression of a disease.

The term “combined product” refers to a fixed or non-fixed combination in the form of a dose unit or a kit of parts for combined administration, in which two or more therapeutic agents may be administered independently at the same time or separately within a certain time interval, especially when such a time interval allows various therapeutic agents in the combined product to exhibit collaborative effects, such as synergistic effects. The term “fixed combination” means that the TNFR2 binding molecule of the present invention and a combination partner (e.g., other therapeutic agents, such as anti-PD-1 antibodies or anti-PD-L1 antibodies) are administered simultaneously to a patient in the form of a single entity or dose. The term “non-fixed combination” means that the TNFR2 binding molecule of the present invention and a combination partner (e.g., other therapeutic agents, such as anti-PD-1 antibodies or anti-PD-L1 antibodies) are administered to a patient simultaneously, concurrently, or sequentially as separate entities, without specific time limits, wherein such administration provides therapeutically effective levels of both therapeutic agents in the patient. The latter also applies to a cocktail therapy, for example, the administration of three or more therapeutic agents. In a preferred embodiment, the pharmaceutical combination is a non-fixed combination.

The term “combination therapy” or “combined therapy” refers to the administration of two or more therapeutic agents to treat cancers as described in the present disclosure. Such an administration involves co-administration of these therapeutic agents in a substantially simultaneous manner, for example, in a single capsule having a fixed ratio of active ingredients. Alternatively, such an administration involves co-administration or separate or sequential administration of the individual active ingredients in several or in separate containers (such as tablets, capsules, powders and liquids). Powders and/or liquids can be reconstituted or diluted to the desired dose prior to administration. In some embodiments, the administration also includes administering each type of therapeutic agent at approximately the same time, or at different times in a sequential manner. In either case, the treatment regimen would provide for the beneficial effects of the pharmaceutical composition in treating the disorders or conditions described herein.

The term “vector” when used herein refers to a nucleic acid molecule capable of propagating another nucleic acid to which it is linked. The term includes the vector as a self-replicating nucleic acid structure and the vector incorporated into the genome of a host cell into which it has been introduced. Some vectors are capable of directing the expression of nucleic acids to which they are operably linked. Such vectors are referred to herein as “expression vectors”.

The term “host cell” refers to a cell into which an exogenous polynucleotide is introduced, including the progeny of such cells. Host cells include “transformant” and “transformed cell”, which include primary transformed cells and progeny derived therefrom regardless of the number of passages. The progeny may be not completely identical to a parent cell in terms of nucleic acid content, but may contain mutations. The mutant progeny that has the same function or biological activity screened or selected in the original transformed cell is included herein. Host cells are any type of cellular system that can be used to produce the antibody molecule of the present invention, including eukaryotic cells such as mammalian cells, insect cells and yeast cells; and prokaryotic cells, for example, E. coli cells. Host cells include cultured cells, and also include cells inside transgenic animals, transgenic plants or cultured plant tissues or animal tissues.

The “subject/patient sample” refers to a collection of cells, tissues or body fluids obtained from a patient or subject. The source of a tissue or cell sample may be a solid tissue, such as from a fresh, frozen and/or preserved organ or tissue sample, or a biopsy sample or a puncture sample; blood or any component of blood; body fluids, such as cerebrospinal fluid, amniotic fluid (liquor amnii), peritoneal fluid (ascites) or interstitial fluid; and cells from any time of pregnancy or development of a subject. Tissue samples may contain compounds that are not naturally intermixed with tissues in nature, such as preservatives, anticoagulants, buffers, fixatives, nutrients and antibiotics. Examples of the tumor samples herein include, but are not limited to, tumor biopsies, fine needle aspirates, bronchial lavage fluid, pleural fluid (hydrothorax), sputum, urine, surgical specimens, circulating tumor cells, blood serum, blood plasma, circulating plasma proteins, ascites, primary cell cultures or cell lines derived from tumors or exhibiting tumor-like properties, and preserved tumor samples such as formalin-fixed, paraffin-embedded tumor samples or frozen tumor samples.

The term “package insert” is used to refer to the instructions for use typically included in commercial packages of therapeutic products that contain information regarding indications, usage, dosage, administration, combination therapies, contraindications and/or warnings involving the use of such therapeutic products.

### II. TNFR2 Binding Molecule of the Present Invention

The TNFR2 binding molecule of the present invention contains at least one single domain antibody (sdAb) moiety that specifically binds to TNFR2, wherein the sdAb moiety contains, from the N-terminus to the C-terminus, three complementarity determining regions, respectively CDR1, CDR2 and CDR3, wherein:
(a) CDR1 contains an amino acid sequence of SEQ ID NO: 3, or a variant with 1 or 2 amino acid changes in the amino acid sequence of SEQ ID NO: 3,
(b) CDR2 contains an amino acid sequence of SEQ ID NO: 4, or a variant with 1 or 2 amino acid changes in the amino acid sequence of SEQ ID NO: 4, and
(c) CDR3 contains an amino acid sequence of SEQ ID NO: 5, or a variant with 1 or 2 amino acid changes in the amino acid sequence of SEQ ID NO: 5,
wherein the amino acid change is an addition, deletion or substitution of an amino acid, and the binding molecule containing the above-mentioned change at least retains the binding ability to TNFR2.

In some embodiments, the TNFR2 binding molecule of the present invention binds to mammalian TNFR2, such as human TNFR2.

In some embodiments, the TNFR2 binding molecule of the present invention has one or more of the following properties:
(1) binding to human TNFR2 with high affinity, for example, the EC₅₀ for the binding between the TNFR2 binding molecule and cell surface TNFR2 is about 0.01 μg/mL to about 1 μg/mL, such as about 0.1 μg/ml to about 0.6 μg/ml;
(2) not substantially blocking the binding of TNFα to TNFR2;
(3) inhibiting the TNFR2 signaling pathway, for example, signaling mediated by TNFR2 is inhibited in TNFR2-expressing cells such as Treg cells (e.g., Treg cells with high expression of CD25), myeloid-derived suppressing cells (MDSCs) and/or TNFR2+ cancer cells;
(4) not substantially affecting the proliferation of Treg cells in PBMCs;
(5) inhibiting tumor growth in vivo.

In some embodiments, the TNFR2 binding molecule of the present invention suppresses the proliferation of Treg cells and/or directly kills Treg cells by binding to and inactivating TNFR2 on the surfaces of the Treg cells (for example, thereby reducing the number of Treg cells in a population of cells by at least about 50%, 55%, 60%, 65%, 70%, 75%, 80%, 85%, 90%, 95%, 96%, 97%, 98%, 99% or 100% relative to the number of Treg cells in a population of cells not exposed to the TNFR2 binding molecule of the present invention).

In some embodiments, the TNFR2 binding molecule of the present invention suppresses the proliferation of MDSCs and/or directly kills MDSCs by binding to and inactivating TNFR2 on the surfaces of the MDSCs (for example, thereby reducing the number of MDSCs in a population of cells by at least about 50%, 55%, 60%, 65%, 70%, 75%, 80%, 85%, 90%, 95%, 96%, 97%, 98%, 99% or 100% relative to the number of MDSCs in a population of cells not exposed to the TNFR2 binding molecule of the present invention).

In some embodiments, the TNFR2 binding molecule of the present invention suppresses the proliferation of cancer cells expressing TNFR2 and/or kills the cancer cells (for example, thereby reducing the number of cancer cells expressing TNFR2 in a population of cells by at least about 50%, 55%, 60%, 65%, 70%, 75%, 80%, 85%, 90%, 95%, 96%, 97%, 98%, 99% or 100% relative to the number of cancer cells expressing TNFR2 in a population of cells not exposed to the TNFR2 binding molecule of the present invention). The cancer cells are, for example, cancer cells from bone cancer, blood cancer, lung cancer, hepatic cancer, pancreatic cancer, esophagus cancer, skin cancer, head and neck cancer, cutaneous or intraocular melanoma, uterine cancer, ovarian cancer, rectal cancer, cancer of the anal region, gastric cancer, colon cancer, breast cancer, prostate cancer, uterine cancer, cancers of sexual organs and reproductive organs, Hodgkin's disease, esophageal cancer, small intestine cancer, cancers of endocrine system, thyroid cancer, parathyroid carcinoma, adrenal cancer, soft tissue sarcomas, bladder cancer, renal cancer, renal cell carcinoma, renal pelvis cancer, tumors of central nervous system (CNS), neuroectodermal cancer, spinal axis tumor, glioma, meningioma, and pituitary adenoma.

In some embodiments, the TNFR2 binding molecule of the present invention reduces the expression of TNFR2 in Treg cells or cancer cells (such as TNFR2+ cancer cells), and/or reduce the secretion of soluble TNFR2 in Treg cells or cancer cells (such as TNFR2+ cancer cells).

In some embodiments, the TNFR2 binding molecule of the present invention is unable to block the binding of TNFα to TNFR2, but is capable of extremely well inhibiting TNFα-TNFR2 signaling pathway-induced cell necrosis. Therefore, the binding epitope of the TNFR2 binding molecule of the present invention is very likely to be located in a domain of a TNFR2 transmembrane protein that is proximal to the cell membrane end.

In some embodiments, the sdAb moiety of the TNFR2 binding molecule of the present invention contains CDR1, CDR2 and CDR3 selected from any one of the following groups:
(a) CDR1 containing an amino acid sequence of SEQ ID NO: 3; CDR2 containing an amino acid sequence of SEQ ID NO: 4; and CDR3 containing an amino acid sequence of SEQ ID NO: 5;
(b) CDR1 containing an amino acid sequence of SEQ ID NO: 10; CDR2 containing an amino acid sequence of SEQ ID NO: 11; and CDR3 containing an amino acid sequence of SEQ ID NO: 12;
(c) CDR1 containing an amino acid sequence of SEQ ID NO: 14; CDR2 containing an amino acid sequence of SEQ ID NO: 15; and CDR3 containing an amino acid sequence of SEQ ID NO: 16;
(d) CDR1 containing an amino acid sequence of SEQ ID NO: 18; CDR2 containing an amino acid sequence of SEQ ID NO: 19; and CDR3 containing an amino acid sequence of SEQ ID NO: 20;
(e) CDR1 containing an amino acid sequence of SEQ ID NO: 22; CDR2 containing an amino acid sequence of SEQ ID NO: 23; and CDR3 containing an amino acid sequence of SEQ ID NO: 24;
(f) CDR1 containing an amino acid sequence of SEQ ID NO: 26; CDR2 containing an amino acid sequence of SEQ ID NO: 27; and CDR3 containing an amino acid sequence of SEQ ID NO: 28;
(g) CDR1 containing an amino acid sequence of SEQ ID NO: 30; CDR2 containing an amino acid sequence of SEQ ID NO: 31; and CDR3 containing an amino acid sequence of SEQ ID NO: 32;
(h) CDR1 containing an amino acid sequence of SEQ ID NO: 34; CDR2 containing an amino acid sequence of SEQ ID NO: 35; and CDR3 containing an amino acid sequence of SEQ ID NO: 36;
(i) CDR1 containing an amino acid sequence of SEQ ID NO: 38; CDR2 containing an amino acid sequence of SEQ ID NO: 39; and CDR3 containing an amino acid sequence of SEQ ID NO: 40;
(j) CDR1 containing an amino acid sequence of SEQ ID NO: 42; CDR2 containing an amino acid sequence of SEQ ID NO: 43; and CDR3 containing an amino acid sequence of SEQ ID NO: 44;
(k) CDR1 containing an amino acid sequence of SEQ ID NO: 46; CDR2 containing an amino acid sequence of SEQ ID NO: 47; and CDR3 containing an amino acid sequence of SEQ ID NO: 48;
(l) CDR1 containing an amino acid sequence of SEQ ID NO: 50; CDR2 containing an amino acid sequence of SEQ ID NO: 51; and CDR3 containing an amino acid sequence of SEQ ID NO: 52.

In some embodiments, the TNFR2 binding molecule of the present invention contains at least one single domain antibody (sdAb) moiety that specifically binds to TNFR2, wherein the sdAb moiety is VHH. In some embodiments, the VHH contains or consists of the following sequences:
(i) an amino acid sequence selected from any one of amino acid sequences of SEQ ID NOs: 6, 7, 8, 13, 17, 21, 25, 29, 33, 37, 41, 45, 49 and 53;
(ii) an amino acid sequence having at least 85%, 90%, 91%, 92%, 93%, 94%, 95%, 96%, 97%, 98% or 99% identity to the amino acid sequence selected from any one of amino acid sequences of SEQ ID NOs: 6, 7, 8, 13, 17, 21, 25, 29, 33, 37, 41, 45, 49 and 53; or
(iii) an amino acid sequence having one or more (preferably no more than 10, more preferably no more than 6, 5, 4, 3, 2 or 1) amino acid changes (preferably amino acid substitution, more preferably conservative amino acid substitution) compared with the amino acid sequence selected from any one of amino acid sequences of SEQ ID NOs: 6, 7, 8, 13, 17, 21, 25, 29, 33, 37, 41, 45, 49 and 53, wherein preferably, the amino acid changes do not occur in the CDR region.

In some embodiments, the TNFR2 binding molecule of the present invention contains at least one single domain antibody (sdAb) moiety that specifically binds to TNFR2, wherein the sdAb moiety is partially humanized or fully humanized VHH or chimeric VHH. Compared with camelid VHHs, the partially humanized or fully humanized VHHs or chimeric VHHs of the present invention have reduced human anti-camelid antibody responses to the human body, improving the safety of antibody application; and are affinity-matured VHHs.

In some embodiments, the TNFR2 binding molecule of the present invention is linked to the Fc region of an immunoglobulin at the N-terminus or C-terminus of the sdAb moiety of the TNFR2 binding molecule, optionally via an amino acid linker, for example, via an amino acid linker having a length between 1 and 20 amino acids. In some embodiments, at least 90% of the amino acid linkers are glycine and/or serine. In some embodiments, the Fc region is from IgG, such as IgG1, IgG2, IgG3 or IgG4. In some embodiments, the Fc region is from human IgG1. In some embodiments, the Fc region is from human IgG2.

In some embodiments of the present invention, the amino acid change described herein includes amino acid substitution, insertion or deletion. Preferably, the amino acid change described herein is amino acid substitution, preferably a conservative substitution.

In preferred embodiments, the amino acid change of the present invention occurs in a region outside CDR (such as in FR). More preferably, the amino acid change of the present invention occurs in a region outside VHH. In some embodiments, the substitution is a conservative substitution. The conservative substitution refers to the substitution of an amino acid with another amino acid within the same class (see, for example, Watson et al., Molecular Biology of the Gene, 4th Edition, 1987, The Benjamin/Cummings Pub. co., p. 224), such as the substitution of an acidic amino acid with another acidic amino acid, the substitution of a basic amino acid with another basic amino acid, or the substitution of a neutral amino acid with another neutral amino acid.

In certain embodiments, the TNFR2 binding molecule provided herein is altered to increase or decrease the extent to which it is glycosylated. Addition or deletion of glycosylation sites to the TNFR2 binding molecule can be conveniently accomplished by altering the amino acid sequence to create or remove one or more glycosylation sites. When the TNFR2 binding molecule contains an Fc region, the saccharides linked to the Fc region can be changed. In some applications, it may be useful to remove unwanted glycosylation site modifications, for example to remove fucose modules to increase the antibody-dependent cell-mediated cytotoxicity (ADCC) function (see Shield et al., (2002) JBC 277:26733). In other applications, galactosylation modifications may be performed to modulate complement-dependent cytotoxicity (CDC). In certain embodiments, one or more amino acid modifications can be introduced into the Fc regions of the TNFR2 binding molecule provided herein, thereby creating Fc region variants in order to enhance, for example, the effectiveness of the TNFR2 binding molecule of the present invention in treating cancers.

In some embodiments, the TNFR2 binding molecule of the present invention is a bispecific or multispecific antibody molecule, wherein the bispecific antibody molecule specifically binds to a TNFR2 molecule and a second target protein. In one embodiment, the second target protein can be any antigen of interest, which is selected from, for example, a tumor antigen (such as a tumor associated antigen and a tumor specific antigen), an immunoregulatory receptor, and an immune checkpoint molecule. As used herein, the “tumor associated antigen” refers to an antigen that is highly expressed in tumor cells, but is also expressed in healthy cells at a lower level. As used herein, the “tumor specific antigen” refers to an antigen that is specifically expressed in tumor cells, but is rarely expressed in healthy cells. Non-limiting examples of the tumor antigens may include CD19, CD20, EGFR, GPC3, HER-2 and FOLR1. Non-limiting examples of the immune checkpoint molecules may include CTLA-4, LAG-3 and TIM-3. Immunoregulatory receptors may include, for example, immune activating receptors (such as CD27, CD137, CD40, GITR and OX40) and immunosuppressive receptors (such as BTLA, CTLA4 and LAG-3). The multispecific antibody molecule may, for example, be a trispecific antibody molecule having a first binding specificity for TNFR2 and second and third binding specificities for one or more of the following molecules: EGFR, GPC3, 4-1BB, OX40 or LAG-3.

### III. Nucleic Acid and Host Cell Containing the Nucleic Acid of the Present Invention

In one aspect, the present invention provides a nucleic acid encoding any of the above TNFR2 binding molecules or the fragments thereof or any of the chains thereof. In one embodiment, provided is a vector containing the nucleic acid. In one embodiment, the vector is an expression vector, for example, a eukaryotic expression vector. In some embodiments, the vector is a viral vector, such as an adenoviral vector, a retroviral vector, a poxviral vector, an adeno-associated viral vector, a baculoviral vector, a herpes simplex viral vector, or a vaccinia viral vector.

In one embodiment, provided is a host cell containing the nucleic acid or the vector. In one embodiment, the host cell is a eukaryotic cell. In another embodiment, the host cell is selected from a yeast cell, a mammalian cell (such as a CHO cell or an HEK293 cell), or other cells suitable for the preparation of antibodies or antigen binding fragments thereof. In another embodiment, the host cell is a prokaryotic cell.

In one embodiment, provided are one or more vectors containing the nucleic acid. In one embodiment, the vector is an expression vector, for example, a eukaryotic expression vector. Vectors include, but are not limited to, viruses, plasmids, cosmids, λ phages or yeast artificial chromosomes (YAC). In one embodiment, the vector is a pcDNA3.4-TOPO vector.

Once an expression vector or a DNA sequence has been prepared for expression, the expression vector can be transfected or introduced into a suitable host cell. Various techniques, such as protoplast fusion, calcium phosphate precipitation, electroporation, retroviral transduction, viral transfection, gene gun, lipid-based transfection or other conventional techniques, can be used to achieve this purpose. In the case of protoplast fusion, cells are grown in a culture medium and screened for appropriate activity. Methods and conditions for culturing the resulting transfected cells and for recovering the resulting antibody molecules are known to a person skilled in the art and can be varied or optimized on the basis of the present description and methods known in the prior art, depending on the particular expression vector and mammalian host cell used.

Additionally, cells that have stably incorporated DNA into their chromosomes can be selected by introducing one or more markers that allow selection of transfected host cells. Markers may, for example, provide prototrophy, biocidal resistance (such as antibiotics) or resistance to heavy metals (such as copper) to auxotrophic hosts. A selectable marker gene can be directly linked to a DNA sequence to be expressed or introduced into the same cell by co-transformation. Additional elements may also be required for optimal synthesis of mRNA. These elements may include splicing signals, as well as transcription promoters, enhancers and termination signals.

In one embodiment, provided is a host cell containing the polynucleotide of the present invention. In some embodiments, provided is a host cell containing the expression vector of the present invention. In some embodiments, the host cell is selected from a yeast cell, a mammalian cell or other cells suitable for the preparation of antibodies. Suitable host cells include prokaryotic microorganisms, such as E. coli. Host cells can also be eukaryotic microorganisms such as filamentous fungi or yeasts, or various eukaryotic cells such as insect cells. Vertebrate cells can also be used as hosts. For example, mammalian cell lines engineered to be adapted to grow in suspension can be used. Examples of useful mammalian host cell lines include monkey kidney CV1 line transformed by SV40 (COS-7); human embryonic kidney lines (HEK293 or 293F cells), 293 cells, baby hamster kidney cells (BHK), monkey kidney cells (CV1), African green monkey kidney cells (VERO-76), human cervical cancer cells (HELA), canine kidney cells (MDCK), Buffalo rat liver cells (BRL 3A), human lung cells (W138), human liver cells (HepG2), Chinese hamster ovary cells (CHO cells), CHO-S cells, NSO cells, and myeloma cell lines such as Y0, NS0, P3X63 and Sp2/0. For a review of mammalian host cell lines suitable for protein production, see, for example, Yazaki and Wu, Methods in Molecular Biology, Vol. 248 (ed. B. K. C. Lo, Humana Press, Totowa, NJ), pp. 255-268 (2003). In a preferred embodiment, the host cell is a CHO cell or an HEK293 cell.

### IV. Production and Purification of TNFR2 Binding Molecule of the Present Invention

In one embodiment, the present invention provides a method for preparing a TNFR2 binding molecule, wherein the method comprises culturing a host cell containing a nucleic acid encoding the TNFR2 binding molecule or an expression vector containing the nucleic acid under conditions suitable for expressing a nucleic acid encoding the TNFR2 binding molecule, and optionally isolating the TNFR2 binding molecule. In a certain embodiment, the method further comprises recovering the TNFR2 binding molecule from the host cell (or a host cell culture medium).

For recombinant production of the TNFR2 binding molecule of the present invention, a nucleic acid encoding the TNFR2 binding molecule of the present invention is first isolated and inserted into a vector for further cloning and/or expression in a host cell. Such nucleic acids are readily isolated and sequenced using a conventional procedure, for example, using an oligonucleotide probe that is capable of specifically binding to a nucleic acid encoding the TNFR2 binding molecule of the present invention.

The TNFR2 binding molecule of the present invention prepared as described herein can be purified by techniques known in the prior art, such as high-performance liquid chromatography, ion exchange chromatography, gel electrophoresis, affinity chromatography and size exclusion chromatography. The actual conditions used to purify a particular protein also depend on factors such as net charge, hydrophobicity and hydrophilicity, which will be apparent to a person skilled in the art. The purity of the TNFR2 binding molecule of the present invention can be determined by any of a variety of well-known analytical methods, including size exclusion chromatography, gel electrophoresis, high-performance liquid chromatography, etc.

### V. Activity Assay of TNFR2 Binding Molecule of the Present Invention

The TNFR2 binding molecule provided herein can be identified, screened or characterized for its physical/chemical properties and/or biological activities by a variety of assays known in the art. In one aspect, the TNFR2 binding molecule of the present invention is tested for its binding activity to an antigen, for example, by known methods such as FACS, ELISA or Western blotting. The binding to TNFR2 can be determined using methods known in the art, and exemplary methods are disclosed herein. In some embodiments, FACS is used to determine the binding of the TNFR2 binding molecule of the present invention to cell surface TNFR2 (such as human TNFR2).

Cells for use in any of the above in-vitro assays include cell lines that naturally expressing TNFR2 or are engineered to express TNFR2. The cell line engineered to express TNFR2 is a cell line that normally does not express TNFR2, but expresses TNFR2 upon transfection of DNA encoding TNFR2 into the cell.

### VI. Pharmaceutical Composition and Pharmaceutical Preparation

In some embodiments, the present invention provides a composition containing any of the TNFR2 binding molecules described herein, preferably the composition is a pharmaceutical composition. In one embodiment, the composition further contains a pharmaceutical auxiliary material. In one embodiment, the composition (such as a pharmaceutical composition) contains a combination of the TNFR2 binding molecule of the present invention, and one or more other therapeutic agents (such as chemotherapeutic agents, cytotoxic agents, other antibodies, small molecule drugs or immunomodulators, for example, anti-PD-1 antibodies or anti-PD-L1 antibodies).

In some embodiments, the composition is used for treating tumors. In some embodiments, the tumors are cancers.

The present invention also includes a composition (including a pharmaceutical composition or a pharmaceutical preparation) containing a TNFR2 binding molecule and/or a composition (including a pharmaceutical composition or a pharmaceutical preparation) containing a polynucleotide encoding a TNFR2 binding molecule. These compositions may also contain suitable pharmaceutical auxiliary materials, such as pharmaceutical carriers and pharmaceutical excipients known in the art, including buffers.

As used herein, the “pharmaceutical carrier” includes any and all solvents, dispersion media, isotonic agents, absorption delaying agents, etc. that are physiologically compatible. Pharmaceutical carriers suitable for use in the present invention can be sterile liquids, such as water and oils, including those of petroleum, animal, vegetable or synthetic origin, such as peanut oil, soybean oil, mineral oil and sesame oil. Water is a preferred carrier when the pharmaceutical composition is administered intravenously. Saline solutions, aqueous dextrose and glycerol solutions can also be used as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, etc. For using excipients and the use of excipients, reference can also be made to “Handbook of Pharmaceutical Excipients”, fifth edition, R. C. Rowe, P. J. Seskey and S. C. Owen, Pharmaceutical Press, London, Chicago. The composition, if desired, can also contain small amounts of wetting agents or emulsifying agents, or pH buffers. These compositions can be in the form of solutions, suspensions, emulsions, tablets, pills, capsules, powders, sustained-release preparations, etc.

The pharmaceutical preparation containing the TNFR2 binding molecule described herein can be prepared by mixing the TNFR2 binding molecule of the present invention having the desired purity with one or more optional pharmaceutical auxiliary materials (Remington's Pharmaceutical Sciences, 16th edition, Osol, A. ed. (1980)), preferably in the form of a lyophilized preparation or an aqueous solution.

The pharmaceutical composition or preparation of the present invention may also contain more than one active ingredient, which is required for the particular indication being treated, preferably those having complementary activities that do not adversely affect each other. For example, it is desirable to also provide other anti-cancer active ingredients, such as chemotherapeutic agents, cytotoxic agents, other antibodies, small molecule drugs or immunomodulators, for example, anti-PD-1 antibodies, anti-PD-L1 antibodies, etc. Such active ingredients are suitably present in combination in an amount effective for the intended use.

Sustained-release preparations can be prepared. Suitable examples of the sustained-release preparations include semi-permeable matrices of solid hydrophobic polymers containing the TNFR2 binding molecule of the present invention, which matrices are in the form of shaped articles such as films or microcapsules.

### VII. Combined Product or Kit

In some embodiments, the present invention also provides a combined product containing the TNFR2 binding molecule of the present invention or the antigen binding fragment thereof, and one or more other therapeutic agents (such as chemotherapeutic agents, other antibodies, cytotoxic agents, small molecule drugs or immunomodulators). In some embodiments, other antibodies are, for example, anti-PD-1 antibodies and anti-PD-L1 antibodies.

In some embodiments, the combined product is used for treating tumors. In some embodiments, the tumors are cancers, etc.

In some embodiments, two or more ingredients of the combined product may be sequentially, separately or simultaneously administered in combination to a subject.

In some embodiments, the present invention also provides a kit containing the TNFR2 binding molecule, the pharmaceutical composition or the combined product of the present invention, and optionally a package insert directing administration.

In some embodiments, the present invention also provides a pharmaceutical product containing the TNFR2 binding molecule, the pharmaceutical composition or the combined product of the present invention, and optionally further containing a package insert directing administration.

### VIII. Use of TNFR2 Binding Molecule of the Present Invention

In one aspect, the present invention relates to a method for treating a disease associated with TNFR2 in a subject, wherein the method comprises administering to the subject a therapeutically effective amount of the TNFR2 binding molecule disclosed herein, or a pharmaceutical composition or a combined product containing the TNFR2 binding molecule.

In some embodiments, the present invention relates to a method for treating a cancer that expresses or overexpresses TNFR2 in a subject, wherein the method comprises administering to the subject a therapeutically effective amount of the TNFR2 binding molecule disclosed herein, or a pharmaceutical composition or a combined product containing the TNFR2 binding molecule. In some embodiments, the cancer that expresses or overexpresses TNFR2 is, for example, bone cancer, blood cancer, lung cancer, hepatic cancer, pancreatic cancer, esophagus cancer, skin cancer, head and neck cancer, cutaneous or intraocular melanoma, uterine cancer, ovarian cancer, rectal cancer, cancer of the anal region, gastric cancer, colon cancer, breast cancer, prostate cancer, uterine cancer, cancers of sexual organs and reproductive organs, Hodgkin's disease, esophageal cancer, small intestine cancer, cancers of endocrine system, thyroid cancer, parathyroid carcinoma, adrenal cancer, soft tissue sarcomas, bladder cancer, renal cancer, renal cell carcinoma, renal pelvis cancer, tumors of central nervous system (CNS), neuroectodermal cancer, spinal axis tumor, glioma, meningioma, and pituitary adenoma.

The subject may be a mammal, for example, a primate, preferably a higher primate, for example, human (such as a patient suffering from or at risk of suffering from the disease described herein). In one embodiment, the subject suffers from or is at risk of suffering from the disease described herein (such as the tumor described herein). In certain embodiments, the subject receives or has received other therapies such as chemotherapy and/or radiation therapy.

In some embodiments, the cancers described herein include, but are not limited to, solid tumors, blood cancers, soft tissue tumors and metastatic lesions.

In some embodiments, the therapies described herein further comprise administering to a subject or individual the TNFR2 binding molecule or the pharmaceutical composition or the combined product disclosed herein in combination with one or more other therapies, such as therapeutic methods and/or other therapeutic agents.

In some embodiments, the therapeutic methods include surgery (such as tumor resection); radiation therapy (such as external beam therapy, which involves three-dimensional conformal radiation therapy in which an area of irradiation is designed), localized irradiation (such as irradiation directed at a preselected target or organ, or focused irradiation), etc. The focused irradiation may be selected from stereotactic radiosurgery, fractionated stereotactic radiosurgery and intensity modulated radiotherapy. The focused irradiation may have a radiation source selected from a particle beam (proton), cobalt-60 (photon) and a linear accelerator (X-ray), for example, as described in WO 2012177624A1.

Radiation therapy can be administered by one of or a combination of several methods including, but not limited to, external beam therapy, internal radiation therapy, implant irradiation, stereotactic radiosurgery, systemic radiation therapy, radiotherapy and permanent or transient interstitial brachytherapy.

In some embodiments, the therapeutic agents are selected from chemotherapeutic agents and other antibodies.

Exemplary other antibodies include, but are not limited to, inhibitors of immune checkpoint molecules (such as anti-PD-1, anti-PD-L1, anti-TIM-3, anti-CEACAM, or anti-LAG-3 antibodies); and antibodies that stimulate immune cells (such as agonistic GITR antibodies or CD137 antibodies). Preferably, the other antibodies are selected from anti-PD-1 antibodies and/or anti-PD-L1 antibodies. More preferably, the anti-PD-1 antibodies are Nivolumab from Bristol-Myers Squibb Company (BMS) and Pembrolizumab from Merck; and the anti-PD-L1 antibodies are atezolizumab developed by Roche, avelumab developed cooperatively by Merck KGaA and Pfizer, and durvalumab developed by AstraZeneca.

Combination therapies of the present invention encompass combined administration (in which two or more therapeutic agents are contained in the same preparation or separate preparations) and separate administration. In the case of separate administration, the administration of the TNFR2 binding molecule of the present invention, etc. may be performed prior to, simultaneously with and/or after administration of other therapies.

In one embodiment, the administration of the TNFR2 binding molecule and the administration of other therapies (such as therapeutic methods or therapeutic agents) occur within about one month, or within about one, two or three weeks, or within about 1, 2, 3, 4, 5 or 6 days of each other.

The TNFR2 binding molecule (and the pharmaceutical composition containing same) of the present invention can be administered by any suitable method, including parenteral administration, intrapulmonary administration and intranasal administration, and, if topical treatment is desired, intralesional administration. Parenteral infusion includes intramuscular, intravenous, intraarterial, intraperitoneal or subcutaneous administration. Administration can be performed by any suitable route, for example, by injection, such as intravenous or subcutaneous injection, depending to a certain extent on whether dosing is short-term or long-term. A variety of dosing schedules are encompassed herein, including but not limited to single administration, multiple administrations at multiple time points, bolus administration and pulse infusion.

To prevent or treat diseases, suitable doses of the TNFR2 binding molecule of the present invention (when used alone or in combination with one or more other therapeutic agents) will depend on the type of a disease to be treated, the type of the TNFR2 binding molecule, the severity and progression of the disease, whether the TNFR2 binding molecule is administered for prophylactic or therapeutic purposes, previous treatment, the clinical history and response to the TNFR2 binding molecule of a patient, and the judgment of an attending physician. The TNFR2 binding molecule is suitably administered to a patient in one treatment or a series of treatments. The dose and treatment regimen of the TNFR2 binding molecule can be determined by the skilled person.

It can be understood that any of the above preventions or treatments can be performed by replacing the TNFR2 binding molecule with the composition or the combined product of the present invention.

### IX. Method and Composition for Diagnosis and Detection

In certain embodiments, any of the TNFR2 binding molecules provided herein can be used to detect the presence of TNFR2 in a biological sample. As used herein, the term “detection” includes quantitative or qualitative detection. Exemplary detection methods may involve immunohistochemistry, immunocytochemistry, flow cytometry (such as FACS), magnetic beads complexed with antibody molecules and ELISA. In certain embodiments, the biological sample is blood, blood serum, or other body fluid samples of biological origin. In certain embodiments, the biological sample contains cells or tissues. In some embodiments, the biological sample is from a hyperproliferative or cancerous lesion.

In one embodiment, provided is a TNFR2 binding molecule for use in a diagnosis or detection method. In another aspect, provided is a method for detecting the presence of TNFR2 in a biological sample. In certain embodiments, the method comprises detecting the presence of a TNFR2 protein in a biological sample. In certain embodiments, the TNFR2 is human TNFR2. In certain embodiments, the method comprises contacting the biological sample with the TNFR2 binding molecule as described herein under conditions that allow the TNFR2 binding molecule to bind to TNFR2, and detecting whether a complex is formed between the TNFR2 binding molecule and TNFR2. The formation of the complex indicates the presence of TNFR2. The method may be an in vitro or in vivo method. In one embodiment, the TNFR2 binding molecule is used to select a subject suitable for being treated with the TNFR2 binding molecule, for example, wherein the TNFR2 is a biomarker for selecting the subject.

In one embodiment, the TNFR2 binding molecule of the present invention can be used to diagnose cancers or tumors, for example, to evaluate (such as monitor) the treatment or progression, diagnosis and/or staging of the disease described herein (such as a hyperproliferative or cancerous disease) in a subject. In certain embodiments, provided is a labeled TNFR2 binding molecule. Labels include, but are not limited to, labels or moieties that are detected directly (such as fluorescent labels, chromophore labels, electron-dense labels, chemiluminescent labels and radioactive labels), and moieties that are detected indirectly (such as enzymes or ligands), for example, by an enzymatic reaction or a molecular interaction. Exemplary labels include, but are not limited to, radioisotopes ³²P, ¹⁴C, ¹²⁵I, ³H and ¹³¹I, fluorophores such as rare earth chelates or luciferin and derivatives thereof, rhodamine and derivatives thereof, dansyl, umbelliferone, luceriferase such as firefly luciferase and bacterial luciferase (U.S. Pat. No. 4,737,456A), fluorescein, 2,3-dihydrophthalazinedione, horseradish peroxidase (HR), alkaline phosphatase, β-galactosidase, glucoamylase, lysozyme, carbohydrate oxidase such as glucose oxidase, galactose oxidase and glucose-6-phosphate dehydrogenase, heterocyclic oxidase such as uricase and xanthine oxidase, enzymes that oxidize dye precursors with hydrogen peroxide such as HR, lactoperoxidase or microperoxidase, biotin/avidin, spin labels, phage labels, stable free radicals, etc.

In some embodiments of any of the inventions provided herein, the sample is obtained prior to treatment with the TNFR2 binding molecule. In some embodiments, the sample is obtained after the cancer has metastasized. In some embodiments, the sample is formalin-fixed, paraffin-embedded (FFPE) sample. In some embodiments, the sample is a biopsy (such as a core biopsy), a surgical specimen (such as a specimen from surgical resection) or a fine needle aspirate.

In some embodiments, the TNFR2 is detected prior to treatment, for example, prior to initiation of treatment or prior to a certain treatment after a treatment interval.

In some embodiments, provided is a method for treating tumors, wherein the method comprises: detecting the presence of TNFR2 in a subject (such as a sample) (such as a subject sample containing cancer cells), thereby determining a value of TNFR2; comparing the value of TNFR2 with a control value (such as a value of TNFR2 in a sample of a healthy individual); and if the value of TNFR2 is greater than the control value, administering to the subject a therapeutically effective amount of a TNFR2 binding molecule (such as the TNFR2 binding molecule described herein), optionally in combination with one or more other therapies, thereby treating tumors.

It can be understood that various embodiments described in various sections of the present invention, such as diseases, therapeutic agents, therapeutic methods and administration, are equally applicable to, or may be combined with, embodiments of other sections of the present invention. Embodiments described in various sections of the present invention, such as properties, uses and methods, applicable to a TNFR2 binding molecule are equally applicable to a composition, conjugate, combined product, kit, etc. containing the TNFR2 binding molecule.

### EXAMPLES

The following examples are intended to illustrate the present invention only and therefore should not be construed as limiting the present invention in any way.

### Example 1. Preparation of Ligand of TNFR2, Anti-TNFR2 Positive Control Antibody, and Cell Line Overexpressing TNFR2

### 1.1 Preparation of Ligand of TNFR2

The ligand of TNFR2 used in the example was TNFα. A human TNFα extracellular region (as shown in SEQ ID NO: 1) was synthesized according to the sequence provided by the GeneCards database, the C-terminus of the gene sequence encoding the human TNFα extracellular region as shown in SEQ ID NO: 1 was ligated to a human IgG1 Fc fragment (as shown in SEQ ID NO: 2), and then the resulting ligated product was constructed into a eukaryotic expression vector pcDNA3.4-TOPO (Invitrogen). The obtained expression vector was expressed using ExpiCHO transient expression system (Gibco, A29133), and the obtained supernatant was subjected to 0.22 μm filtration and then purified using a Protein A/G affinity purification method, followed by elution with 100 mM glycinate (pH 3.0) to obtain the TNFα-Fc fusion protein that passed the quality inspection.

### 1.2 Preparation of Anti-TNFR2 Positive Control Antibody

The anti-TNFR2 positive control antibody used in the example was hSBT-002e (hereinafter also referred to as “SBT002e”) and was synthesized according to the sequence disclosed in International Application WO 2017083525A1. A plasmid containing SBT002e light chain gene and a plasmid containing SBT002e heavy chain gene were constructed separately by a molecular cloning method. Expression of SBT002e was performed using ExpiCHO transient expression system, and the obtained supernatant was subjected to 0.22 μm filtration and then purified using a Protein A/G affinity purification method, followed by elution with 100 mM glycinate (pH 3.0) to obtain the positive control antibody SBT002e.

### 1.3 Preparation of Cell Line Overexpressing Human TNFR2

An HEK293 cell line overexpressing human TNFR2 (hereinafter referred to as a huTNFR2-HEK293 cell line) and a Jurkat cell line overexpressing human TNFR2 (hereinafter referred to as a huTNFR2-Jurkat cell line) were constructed.

A DNA fragment encoding human TNFR2 full-length protein (amino acid sequence as shown in SEQ ID NO: 9) was synthesized by gene synthesis techniques (General Biosystems (Anhui) Co., Ltd.) and cloned into an expression vector pLVX-puro (Clontech, Cat #632164). The vector was introduced into E. coli DH5a by transformation, E. coli single clones were picked and sequenced to obtain correct plasmid clones, and the plasmids were extracted and sequenced again for confirmation.

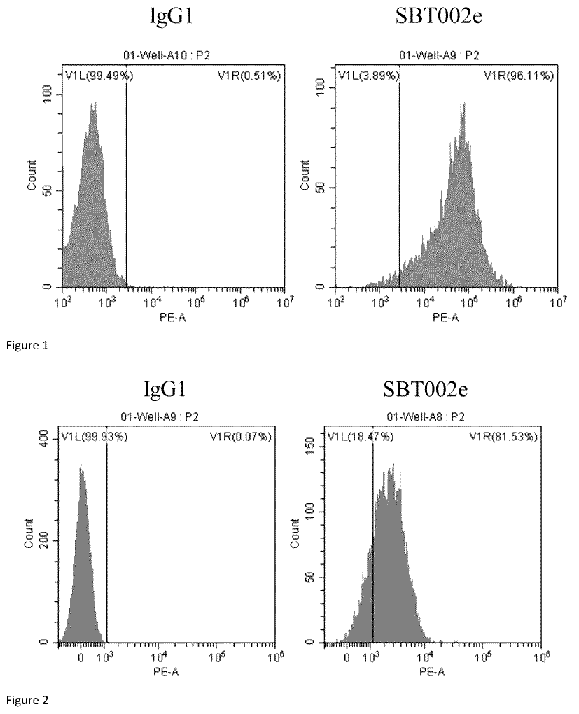

HEK293 cells (ATCC® CRL-1573™) were cultured using a DMEM medium (Cat #11995-665) from Gibco and Jurkat cells (ATCC® TIB-152) were cultured using an RPMI1640 medium (Cat #11875093) from Gibco. One day prior to electroporation, the cells were passaged to 5×10⁵/mL, and the next day, the constructed plasmids were introduced into the cells using an electroporation kit (Cat #MPK10096) from Invitrogen and an electroporator (Invitrogen, Neon™ Transfection System, MP922947). The electroporated cells were transferred to a DMEM medium containing 10% FBS and cultured in a 37° C. cell incubator for 48 h. The cells were then plated in a 96-well plate at 1500-4000 cells/well, puromycin (Gibco, A1113803) was added at a final concentration of 2 μg/mL, the cells were cultured in a 37° C. carbon dioxide incubator, and after 10 days, a DMEM medium containing 2 μg/mL puromycin was supplemented. Cell clones growing in the 96-well plate were picked and transferred to a 24-well culture plate for further scale-up culture. After that, the cell line with successful stable transformation of human TNFR2 was identified by FACS using the control antibody SBT002e. The identification result of the huTNFR2-HEK293 cell line was as shown in FIG. 1, and the identification result of the huTNFR2-Jurkat cell line was as shown in FIG. 2.

### Example 2. Animal Immunization and Immune Titer Determination in Serum

### 2.1 Animal Immunization

Recombinant human TNFR2 (SinoBiological, 10417-H03H) was used as an antigen to immunize 2 alpacas (Nanchang Dajia Technology Co., Ltd.). Each alpaca was immunized with 500 μg of the antigen each time, once every two weeks, for a total of 4 times.

### 2.2 Immune Titer Determination in Serum

After the completion of the alpaca immunization, the alpaca sera were taken for immune titer determination. Immune titer determination was performed by ELISA to determine the binding ability of the immune sera to the recombinant human TNFR2, and to judge the immune effect according to the titer of the antibody that was bound to the antigen.

The specific method was as follows: one day prior to the immune titer determination, the recombinant human TNFR2 was diluted with PBS to a final concentration of 2 μg/mL, to obtain a diluent; 30 μL of the diluent was taken and added to an ELISA plate, and the plate was coated overnight at 4° C.; on the day of the immune titer determination, the coated plate was rinsed three times with PBST, then blocked with PBST containing 5% skim milk powder at room temperature for 2 h, and then rinsed three times with PBST; on another 96-well dilution plate, unimmunized negative sera and the immunized sera were diluted with PBS, with 2000-fold dilution for the first well, followed by 3-fold gradient dilution for the subsequent 7 wells; the diluted sera were added to the first ELISA plate coated with the recombinant human TNFR2 and incubated at room temperature for 1 h; after the plate was washed three times with PBST, anti-IgG (H+L)-HRP (Millipore) was added at a ratio of 1:10000, and the plate was incubated at room temperature for 1 h; after the incubation was completed, the plate was washed six times with PBST, TMB (SurModics, TMBS-1000-01) was added for color development, 2 M HCl was added to stop the reaction according to the color development results, and the OD values were read at a wavelength of OD450 by a microplate reader (Molecular Devices, SpecterMax 190).

The results showed that the serum titer reached 256000 after 4 immunizations and could be used for the construction of an alpaca peripheral blood immune antibody library in the next step.

### Example 3. Construction and Preliminary Screening of Alpaca Immune Library

### 3.1 Library Construction

After the completion of the animal immunization, 50 mL of fresh blood was taken from alpaca, peripheral blood mononuclear cells (PBMCs) were isolated by Ficoll-Paque density gradient separation solution (GE, 17144003S), RNA was extracted from the isolated PBMCs, and the extracted RNA was reverse-transcribed into cDNA by a reverse transcription kit (TaKaRa, 6210A). On the basis of VHH antibody germline genes, a DNA fragment encoding VHH-CH2 was obtained by designing degenerate primers, performing PCR amplification and recovering the PCR product by agarose gel electrophoresis. The recovered DNA fragment product was then used as a template to amplify all VHH genes, and finally the antibody gene fragment of interest was inserted into a phage display vector by double enzyme digestion and ligation. The ligated product was recovered by a recovery kit (Omega, D6492-02) and finally transformed into competent E. coli SS320 (Lucigen, MC1061F) by an electroporator (Bio-Rad, MicroPulser), and the transformed E. coli SS320 was coated on an ampicillin-resistant 2-YT solid plate to construct an anti-human TNFR2 single domain antibody library.

The capacity of the library was determined to be 1.8×10⁹ cfu by gradient dilution plating. The anti-human TNFR2 single domain antibody library was packaged using helper phage M13KO7 (NEB), resulting in a phage library corresponding to the anti-human TNFR2 single domain antibody library.

### 3.2 Screening of Phage Library by Magnetic Bead Method

A biotinylated TNFR2 protein was incubated with avidin-coupled magnetic beads (Thermo fisher, 11205D), such that the TNFR2 protein was bound to the magnetic beads. The magnetic beads that were bound to the TNFR2 antigen and the nanobody-displaying phage library prepared in 3.1 above were incubated at room temperature for 2 h. After the mixture was washed 6-8 times with PBST, the non-specifically adsorbed phages were removed. Trypsin (Gibco) was added and the mixture was mixed gently to uniformity for 20 min, so as to elute the nanobody-displaying phages that were specifically bound to the human TNFR2 protein. The eluted phages were then used to infect SS320 bacteria (Lucigen, MC1061 F) at log-phase, the phage-infected SS320 bacteria were coated on a 50 μg/mL carbenicillin-resistant plate and cultured at 37° C. overnight, and the bacteria were collected the next day. The SS320 bacteria were used for preparing phages for the next round of screening.

### 3.3 Monoclonal Screening

The positive phage libraries in the products from the first and second rounds of the magnetic bead screening were picked for monoclonal screening, respectively. The specific method was as follows: one day prior to the monoclonal screening, a 96-well ELISA plate was coated with recombinant human TNFR2, and the phage supernatant was prepared in the 96-well plate the next day; positive clones against human recombinant TNFR2 (SinoBiological, 10417-H03H) were screened by phage ELISA, and then all the positive clones were picked for sequencing analysis; the bacterial liquid of the positive single clone was inoculated into 50 mL of a 2-YT medium at 1:100, cultured with shaking in a 37° C. constant-temperature shaker for 14 h, and centrifuged at 10000 g for 5 min at room temperature. The bacteria were resuspended in 1 mL of a Tris-HCl buffer containing a benzonase nuclease with PH 9.0, lysed on ice for 30 min, and centrifuged at 10000 g for 10 min at 4° C., and the supernatant was collected to obtain a positive clone lysate.

The prepared positive clone lysate was subjected to ELISA affinity detection. The specific method was as follows: a 96-well ELISA plate was coated with 2 μg/mL recombinant human TNFR2 and incubated overnight at 4° C.; the next day, the well plate was washed 3 times with PBST, 5% skim milk was added, and blocking was performed for 2 h; subsequently, the well plate was washed 3 times with PBST, gradiently diluted positive clone lysates were added, and incubation was performed for 1 h; subsequently, the well plate was washed 3 times with PBST, Rabbit Anti-Camelid-VHH-HRP (Genescript, A01861-200) diluted at 1:8000 was added, and incubation was performed for 1 h; subsequently, the well plate was washed 6 times with PBST, TMB (SurModics, TMBS-1000-01) was added for color development in the dark for 5-10 min, and 2 M HCl was added to stop the reaction according to the color development results; the values at OD450 were read by a microplate reader (Molecular Devices, SpecterMax 190) and fitted by four parameters.

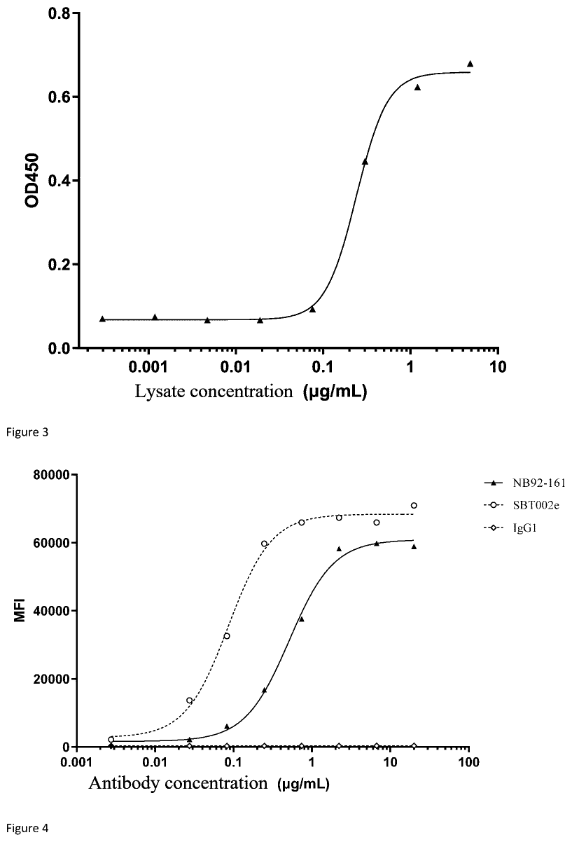

The results were as shown in FIG. 3, indicating that the lysate from the selected positive clone NB92-161 exhibited a dose-dependent binding effect for the binding to the recombinant human TNFR2. The amino acid sequence of VHH of the positive clone NB92-161 was as shown in SEQ ID NO: 6, and the amino acid sequences of CDR1, CDR2 and CDR3 of the positive clone NB92-161 defined by AbM were as shown in SEQ ID NOs: 3-5, respectively.

### Example 4. Production and Expression of VHH-Fc Chimeric Antibody

The VHH obtained by the screening in example 3 was fused with a human IgG1 Fc fragment (as shown in SEQ ID NO: 2), wherein the C-terminus of the VHH gene sequence was ligated to the N-terminus of the human IgG1 Fc fragment gene sequence to construct an expression vector pcDNA3.4-TOPO (Invitrogen) for a VHH-Fc chimeric antibody. Expression was performed using ExpiCHO transient expression system, and the cell culture supernatant expressing a protein of interest was centrifuged at 15000 g (high speed) for 10 min. The resulting supernatant was affinity purified by MabSelect SuRe LX (GE, 17547403), and then the protein of interest was eluted with 100 mM sodium acetate (pH 3.0), followed by neutralization with 1 M Tris-HCl, and finally the resulting protein was exchanged into a PBS buffer through an ultrafiltration concentration tube (Millipore, UFC901096) to obtain the VHH-Fc chimeric antibody that passed the quality inspection.

### Example 5. Evaluation of Affinity Activity of VHH-Fc Chimeric Antibody

The obtained VHH-Fc chimeric antibody was evaluated for affinity activity. The binding activity of the VHH-Fc chimeric antibody to a TNFR2 protein on cells was detected by FACS. The specific method was as follows: the cultured huTNFR2-HEK293 cells were collected and centrifuged at 300 g to remove the supernatant, the cells were resuspended in a prepared FACS buffer (PBS containing 1% BSA) and counted, and the cell suspension density was adjusted to 2×10⁶ cells/mL; the huTNFR2-HEK293 cells were added to a 96-well round-bottom plate at 100 μL/well and centrifuged at 300 g to remove the supernatant; gradiently diluted chimeric antibody NB92-161 (antibody named by clone number) and a control antibody SBT002e were added to each corresponding well of the 96-well round-bottom plate, and the cells were resuspended and then incubated at 4° C. for 30 min; the incubated cell mixed solution was washed 3 times and a PE-labeled anti-human IgG-Fc flow cytometry antibody (Abcam, ab98596) was added; the cells were resuspended and then incubated at 4° C. for 30 min; and the incubated cell mixed solution was washed 3 times, and the cells were resuspended and detected by a flow cytometer (Beckman, CytoFLEX AOO-1-1102).

The detection results of the flow cytometry were as shown in FIG. 4, indicating that the binding activity of the chimeric antibody NB92-161 on the huTNFR2-HEK293 cells was lower than that of the control antibody SBT002e, with the binding EC₅₀ of the NB92-161 of 0.5127 μg/mL and the binding EC₅₀ of the SBT002e of 0.0864 μg/mL, and the affinity at the cellular level of the chimeric antibody NB92-161 against TNFR2 was far lower than that of the control antibody SBT002e.

### Example 6. Verification of Species Cross-Reactivity of VHH-Fc Chimeric Antibody

The obtained VHH-Fc chimeric antibody was verified for species cross-reactivity. The specific method was as follows: a 96-well ELISA plate was coated with 2 μg/mL recombinant cynomolgus monkey TNFR2 (SinoBiological, 90102-C08H) and recombinant mouse TNFR2 (SinoBiological, 50128-M08H), respectively, and incubated overnight at 4° C.; the next day, the well plate was washed 3 times with PBST, 5% skim milk was added, and blocking was performed for 2 h; subsequently, the well plate was washed 3 times with PBST, gradiently diluted chimeric antibody NB92-161 and a control antibody SBT002e were added, and incubation was performed for 1 h; after the incubation was completed, the well plate was washed 3 times with PBST, Goat-Anti-Human-IgG-Fc-HRP (abcam, ab97225) diluted at 1:4000 was added, and incubation was performed for 1 h; subsequently, the well plate was washed 6 times with PBST, TMB (SurModics, TMBS-1000-01) was added for color development in the dark for 5-10 min, and 2 M HCl was added to stop the reaction according to the color development results; the values at OD450 were read by a microplate reader (Molecular Devices, SpecterMax 190) and fitted by four parameters.

The results were as shown in FIG. 5, indicating that the chimeric antibody NB92-161 showed good cross-binding activity with the recombinant cynomolgus monkey TNFR2. In addition, the chimeric antibody NB92-161 had no cross-binding to the recombinant mouse TNFR2, and thus the result was not shown in the figure.

### Example 7. Evaluation of Blocking Activity of VHH-Fc Chimeric Antibody Against the Binding of TNFα to TNFR2

The obtained VHH-Fc chimeric antibody was evaluated for ligand blocking activity. FACS was used to detect whether the VHH-Fc chimeric antibody blocked the binding of TNFα to TNFR2. The specific method was as follows: the cultured huTNFR2-HEK293 cells were collected and centrifuged at 300 g to remove the supernatant, the cells were resuspended in a prepared FACS buffer and counted, and the cell suspension density was adjusted to 2×10⁶ cells/ml; the huTNFR2-HEK293 cells were added to a 96-well round-bottom plate at 100 μL/well and centrifuged at 300 g to remove the supernatant; gradiently diluted chimeric antibody NB92-161 and a control antibody SBT002e were added to each corresponding well of the 96-well round-bottom plate, and the cells were resuspended and then incubated at 4° C. for 30 min; the incubated cell mixed solution was washed 3 times, and then 100 μL of a biotinylated TNFα-Fc fusion protein (prepared in example 1.1 of the present application) diluent (0.1 μg/mL) was added to each corresponding well, and the cells were resuspended and incubated at 4° C. for 30 min; the incubated cell mixed solution was washed 3 times, and then a PE-labeled streptavidin (eBioscience, 12-4317-87) was added, and the resulting mixture was incubated at 4° C. for 30 min; the incubated cell mixed solution was washed 3 times, and then an FACS buffer was added to the wells at 200 μL/well, and the cells were resuspended and detected by a flow cytometer (Beckman, CytoFLEX AOO-1-1102).

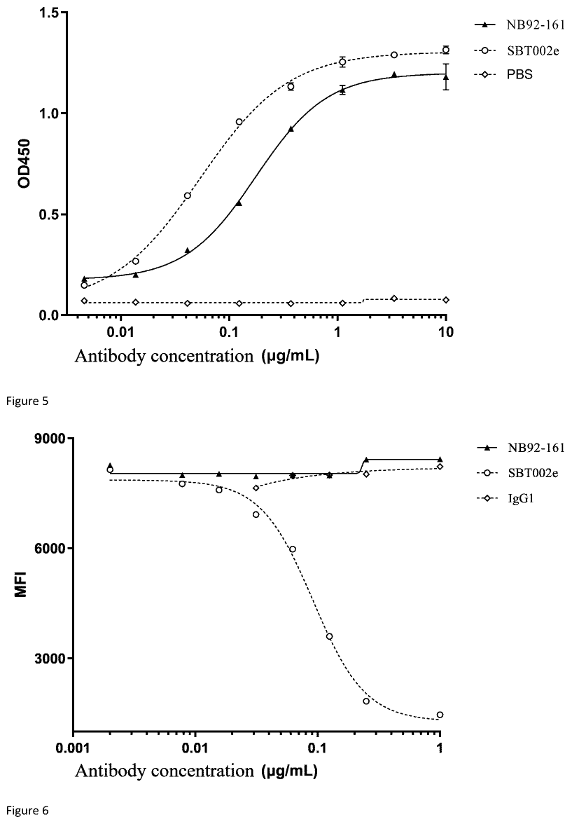

The results that the anti-TNFR2 VHH-Fc chimeric antibody did not substantially block the binding activity of TNFα to TNFR2 on the huTNFR2-HEK293 cells were as shown in FIG. 6. It could be seen from the results that the chimeric antibody NB92-161 did not substantially block the binding of TNFα to TNFR2, whereas the control antibody SBT002e completely blocked the binding of TNFα to TNFR2 with the IC₅₀ of 0.0917 μg/mL.

### Example 8. Evaluation of Inhibitory Activity of VHH-Fc Chimeric Antibody on TNFα-TNFR2 Signaling Pathway-Induced Cell Necrosis

In this example, whether the candidate antibody of the present invention had an inhibitory activity on TNFα-TNFR2 signaling pathway was evaluated by a TNFα-induced cell necrosis experiment. The specific method was as follows: the cultured huTNFR2-Jurkat cells were collected and centrifuged at 300 g to remove the supernatant, the huTNFR2-Jurkat cells were resuspended in a culture medium and counted, and the cell suspension density was adjusted to 2×10⁵ cells/mL; the huTNFR2-Jurkat cells were added to a 96-well round-bottom plate at 50 μL/well; gradiently diluted chimeric antibody NB92-161 and a control antibody SBT002e were added to each corresponding well of the 96-well round-bottom plate at 25 μL/well, and incubated at 37° C. for 2 h; after the incubation was completed, a TNFα-Fc fusion protein (prepared in example 1.1 of the present application) diluent (5 ng/mL) was added to the corresponding wells at 25 μL/well, and incubated at 37° C. for 24 h; after the incubation was completed, Cell-Titer Glo (Promega, G7572) was added at 50 μL/well and incubated for 10 min, and then the plate was placed in a microplate reader (MD, SpectraMax i3x) to detect the fluorescence value.

The results were as shown in FIG. 7, indicating that the chimeric antibody NB92-161 could significantly inhibit the TNFα-induced huTNFR2-Jurkat cell necrosis despite a large difference in the affinity activity for binding to TNFR2 relative to the control antibody SBT002e, and the inhibitory activity was significantly better than that of the control antibody SBT002e, with the ED₅₀ of the chimeric antibody NB92-161 of 0.03390 μg/mL and the ED₅₀ of the control antibody SBT002e of 0.4993 μg/mL. Therefore, the chimeric antibody of the present invention could excellently well inhibit the TNFR2 signaling pathway.

### Example 9. Verification of Efficacy of VHH-Fc Chimeric Antibody in TNFR2 Humanized Mouse Tumor Model

To confirm the activity of the candidate anti-TNFR2 VHH-Fc chimeric antibody in inhibiting tumor growth in vivo, an MC38 tumor-bearing model based on a TNFR2 humanized mouse was established. The specific method was as follows: TNFR2 humanized C57BL/6 mice (Shanghai Model Organisms Center, Inc.) with similar body weights at about 8 weeks of age were selected and divided into a PBS control group, a chimeric antibody NB92-161 group and a positive control antibody SBT002e group, with a total of 3 groups and 5 mice in each group; a mouse colon cancer cell line MC38 (purchased from the Institute of Basic Medical Sciences, Chinese Academy of Medical Sciences) was cultured in vitro, and 1.5×10⁶ MC38 cells were injected subcutaneously into the mice, noted as day 0; the mice in each group were respectively injected with the chimeric antibody NB92-161 at 7.5 mg/kg, the positive control antibody at 15 mg/kg or PBS on the seventh day, and thereafter the administration was performed twice a week for 6 consecutive administrations; the body weights and tumor sizes of the mice were recorded weekly starting on day 7 until the tumors in the PBS control group grew to 1500 mm³; the tumor size was measured by a digital caliper, and the tumor volume was calculated by the formula (L×W²)/2, where L is the longest and W is the shortest of the tumor diameters (mm); the relative tumor volume was equal to the tumor volume at a given time point divided by the tumor volume before the start of the treatment.

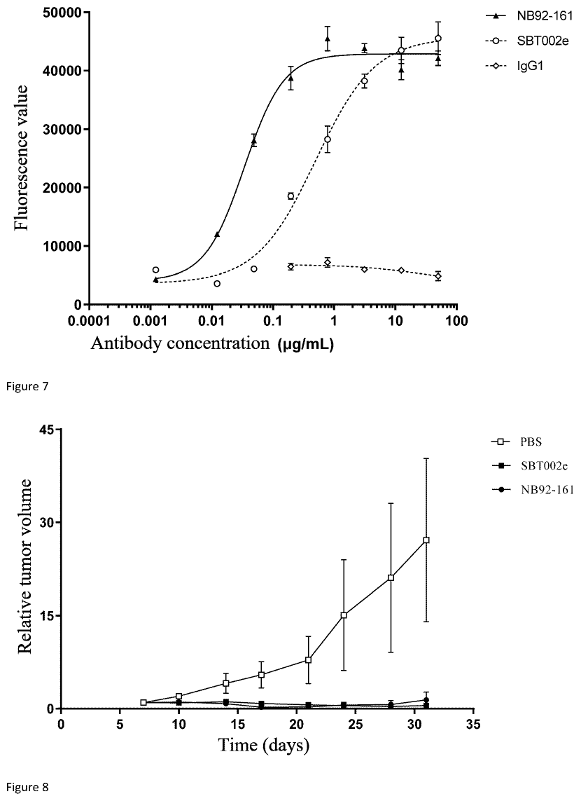

The results were as shown in FIG. 8, indicating that the chimeric antibody NB92-161 showed a strong inhibitory activity on tumor growth and there was no significant difference in the relative tumor volume between the chimeric antibody and the positive control antibody SBT002e.

### Example 10. Humanization Modification of Antibody

To reduce the immunogenicity of an antibody molecule in vivo, the anti-TNFR2 VHH-Fc chimeric antibody NB92-161 was subjected to humanization design. The antibody sequence was aligned with the human antibody germline gene database and 1-3 germline genes that had high homology to each VHH sequence were found, while taking into account the druggability of the germline genes, appropriate germline gene templates were selected for alignment and the number of non-human sequence sites in the VHH framework region was analyzed. Homology modeling was performed on VHH. The homology modeling referred to the nanobody model of the PDB database (http://www.rcsb.org/). Combined with the simulated structural model and non-human sites of VHH, combinatorial backmutation design was performed (while avoiding the introduction of potential post-translational modification sites), and sequences with different degrees of humanization were designed. The VHH amino acid sequences of the humanized antibodies NB92-161-hVH5 and NB92-161-hVH4 obtained after the modification of the anti-TNFR2 VHH-Fc chimeric antibody were as shown in SEQ ID NO: 7 and SEQ ID NO: 8, with degrees of humanization of 95.87% and 94.21%, respectively.

### Example 11. Binding of Humanized Antibody to huTNFR2-HEK293 Cells

To detect the binding activity of the humanized antibody to a human TNFR2 antigen, FACS was used to detect the anti-TNFR2 VHH-Fc chimeric antibody and the corresponding humanized antibody thereof. The specific method was similar to that in example 5.

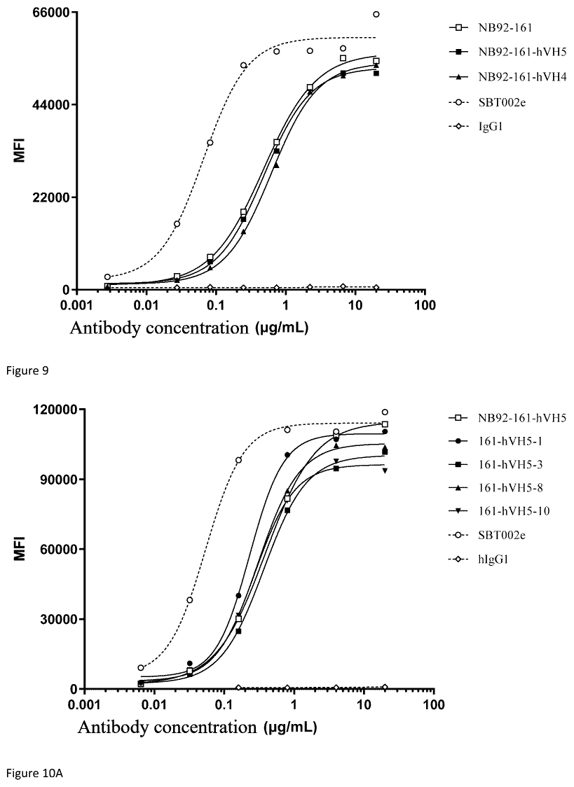

The results were as shown in FIG. 9. It could be seen from FIG. 9 that the binding ability of the humanized antibodies NB92-161-hVH5 and NB92-161-hVH4 to human TNFR2 was comparable to that of the anti-TNFR2 VHH-Fc chimeric antibody NB92-161 (the parent molecule).

### Example 12. Affinity Maturation of Humanized Antibody

In this example, the humanized antibody NB92-161-hVH5 was subjected to affinity maturation to improve the affinity and the biological activity. The affinity maturation was based on the M13 phage display technology, CDR region mutations were introduced using codon-based primers (in the primer synthesis process, a single codon was composed of NNK), and a total of 4 phage display libraries were constructed: library 1 was CDR1+CDR2+CDR3 single-point combined mutation; library 2 was CDR1+CDR2 double-point combined mutation; library 3 was CDR1+CDR3 double-point combined mutation; and library 4 was CDR2+CDR3 double-point combined mutation. The capacities of the libraries were as shown in Table 1.

A single CDR region mutant fragment was obtained by PCR with the humanized antibody NB92-161-hVH5 as a template, a VHH full-length fragment was obtained by Overlapping PCR, the point mutant antibody was ligated into a phage display vector by double enzyme digestion (Hind Ill and Not I) and double-sticky-end ligation, and finally the VHH sequence with a mutation site was transformed into E. coli SS320 by electroporation.

After the 4 constructed libraries were packaged into phages, solid-phase panning was performed. Antigens coated on an immunotube were bound to the phages displaying the VHH full-length fragment, and antibodies with high affinity were subjected to panning by reducing the mass pressure of the coated antigens. After the panning, elution and infection of E. coli SS320, the next cycle of panning was performed. After 2-3 cycles of the panning, single clones were selected for ELISA affinity detection. According to the affinity and sequence analysis, 11 candidate anti-TNFR2 affinity-matured molecules were selected for sample preparation, and the preparation method was detailed in example 4. The variable region amino acid sequence information of the candidate anti-TNFR2 affinity-matured molecules was as shown in Table 2.

TABLE 1
Library design and library capacity

| | | | Theo- | |
|---|---|---|---|---|
| Name | | | retical | Actual |
| of | | | library | library |
| library | Library design | Mutation design | capacity | capacity |
| Library 1 | CDR1 + CDR2 + | Single-point | 9.60E+06 | 1.20E+08 |
| | CDR3 | combined mutation | | |
| Library 2 | CDR1 + CDR2 | Double-point | 1.30E+07 | 4.50E+08 |
| | | combined mutation | | |
| Library 3 | CDR2 + CDR3 | Double-point | 1.60E+07 | 1.01E+08 |
| | | combined mutation | | |
| Library 4 | CDR1 + CDR3 | Double-point | 1.60E+07 | 1.44E+08 |
| | | combined mutation | | |

TABLE 2
Variable region amino acid sequences (SEQ ID NO:) of
candidate anti-TNFR2 affinity-matured molecules

| Name of molecule | HCDR1 | HCDR2 | HCDR3 | VHH |
|---|---|---|---|---|
| 161-hVH5-1 | 10 | 11 | 12 | 13 |
| 161-hVH5-3 | 14 | 15 | 16 | 17 |
| 161-hVH5-8 | 18 | 19 | 20 | 21 |
| 161-hVH5-10 | 22 | 23 | 24 | 25 |
| 161-hVH5-19 | 26 | 27 | 28 | 29 |
| 161-hVH5-22 | 30 | 31 | 32 | 33 |
| 161-hVH5-24 | 34 | 35 | 36 | 37 |
| 161-hVH5-36 | 38 | 39 | 40 | 41 |
| 161-hVH5-37 | 42 | 43 | 44 | 45 |
| 161-hVH5-48 | 46 | 47 | 48 | 49 |
| 161-hVH5-49 | 50 | 51 | 52 | 53 |

### Example 13. Evaluation of Affinity Activity of Affinity-Matured Molecule

To detect the binding activity of the affinity-matured molecule to a human TNFR2 antigen, FACS was used to detect the candidate affinity-matured molecules in this example. For the specific method, see example 5.

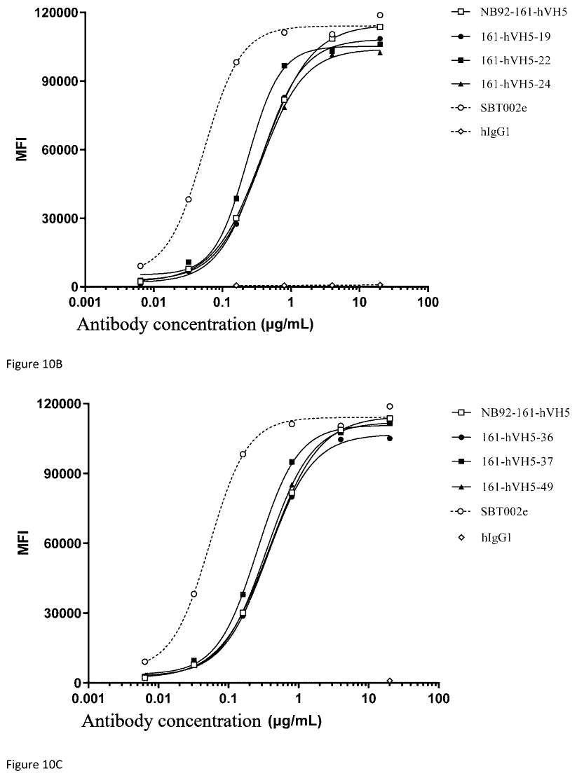

The detection results were as shown in FIGS. 10A-10D. It could be seen from FIGS. 10A-10C that the 10 candidate affinity-matured molecules presented had comparable or superior affinity activity to the parent molecule NB92-161-hVH5. As shown in FIG. 10D and Table 3, the affinity activity of the affinity-matured molecule 161-hVH5-48 was close to that of the control antibody SBT002e and increased by about 5 times compared with that of the parent molecule NB92-161-hVH5. The EC₅₀ values were as shown in Table 3.

TABLE 3
Affinity activity of affinity-matured molecule

| Name of molecule | EC₅₀ (μg/mL) |
|---|---|
| NB92-161-hVH5 | 0.3359 |
| 161-hVH5-48 | 0.0664 |
| SBT002e | 0.0698 |

### Example 14. Evaluation of Blocking Activity of Affinity-Matured Molecule Against Binding of TNFα to TNFR2

To detect whether the affinity-matured molecule had a blocking activity on the binding of TNFα to TNFR2, a ligand blocking activity was evaluated in this example. The specific method was as described in example 7.

The results were as shown in FIG. 11, indicating that the affinity-matured molecule 161-hVH5-48, like the parent molecule NB92-161-hVH5, did not block the binding of TNFα to TNFR2.

### Example 15. Evaluation of Inhibitory Activity of Affinity-Matured Molecule on TNFα-TNFR2 Signaling Pathway-Induced Cell Necrosis

To detect whether the affinity-matured molecule still had a strong inhibitory activity on TNFα-induced TNFR2 signaling pathway, the inhibitory activity was evaluated by a TNFα-induced cell necrosis experiment in this example. The specific method was as described in example 8.

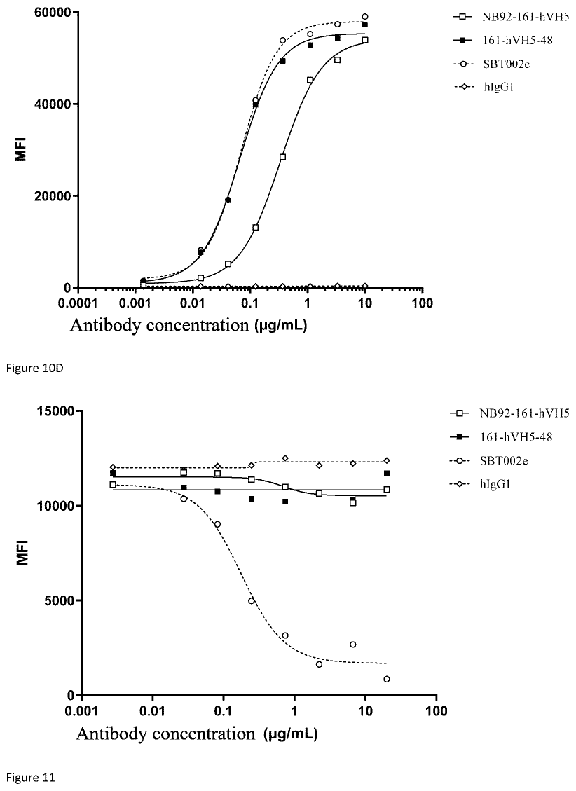

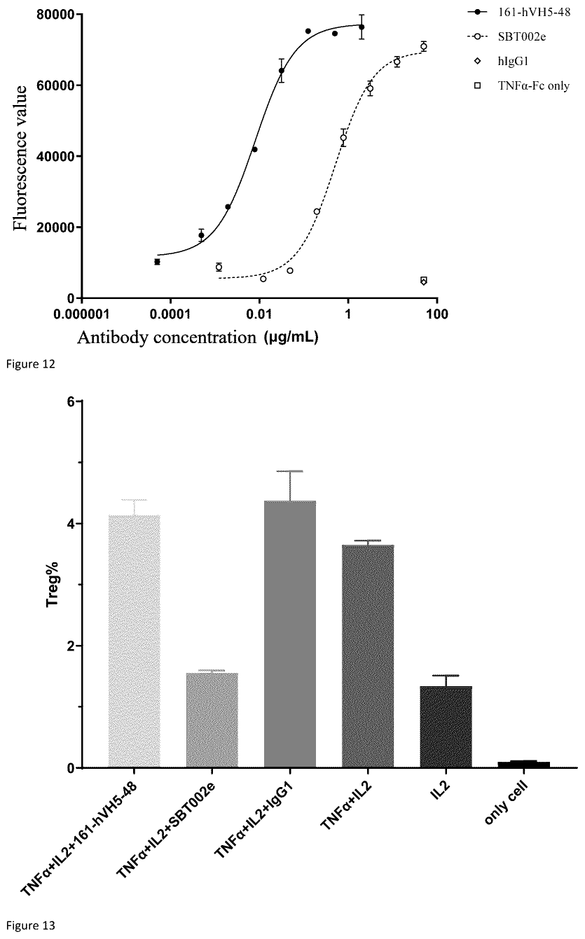

The detection results were as shown in FIG. 12, indicating that the affinity-matured molecule 161-hVH5-48 still had an extremely excellent inhibitory activity, much higher than that of the control antibody SBT002e, with the ED₅₀ of the affinity-matured molecule 161-hVH5-48 of 0.008109 μg/mL and the ED₅₀ of the control antibody SBT002e of 0.5137 μg/mL.

### Example 16. Study on Effect of Affinity-Matured Molecule on Proliferation of Treg Cells in PBMCs

To detect whether the affinity-matured molecule affected the proliferation of Treg cells in PBMCs, a TNFα-induced Treg cell proliferation experiment was used for evaluation in this example. The specific method was as follows: firstly PBMCs were isolated from fresh blood and then further isolated using a CD4⁺ T cell isolation kit (Miltenyi, 130-096-533) to obtain CD4⁺ T cells; the CD4⁺ T cells were collected and centrifuged at 300 g to remove the supernatant, the cells were resuspended in a complete medium and counted, and the cell suspension density was adjusted to 2×10⁶ cells/mL; the CD4⁺ T cells were added to a 96-well round-bottom plate at 100 μL/well, and the affinity-matured molecule 161-hVH5-48 and the control antibody SBT002e prepared using a culture medium containing 400 U/mL IL2 (Novoprotein, CP09) and 40 ng/ml TNFα (Sino Biological, 10602-H01H) were added to each corresponding well of the 96-well round-bottom plate at 100 μL/well and incubated at 37° C. for 72 h; the incubated cell mixed solution was washed 3 times, and then a PE-labeled anti-human CD4 flow cytometry antibody (BioLegend, 357404) and an FITC-labeled anti-human CD25 flow cytometry antibody (BioLegend, 356106) were added, and the cells were resuspended and incubated at 4° C. for 30 min; the incubated cell mixed solution was washed 3 times, and then 4% paraformaldehyde solution was added, and the mixture was fixed at room temperature for 30 min; after washing 3 times with 1× Perm solution, an Alexa Fluor® 647-labeled anti-human Foxp3 flow cytometry antibody (BioLegend, 320114) was added, incubation was performed at room temperature for 1 h; the incubated cell mixed solution was washed 3 times, and the cells were resuspended and detected by a flow cytometer (Beckman, CytoFLEX AOO-1-1102).

The detection results of the flow cytometry were as shown in FIG. 13: the affinity-matured molecule 161-hVH5-48 did not affect the proliferation of Treg cells in PBMCs; while the positive antibody SBT002e could significantly inhibit the proliferation of Treg cells, which might cause unnecessary hematotoxicity.

### Example 17. Evaluation of Tumor Suppression Activity of Affinity-Matured Molecule in Humanized Mice

To detect the tumor suppression ability of the affinity-matured molecule in mice, a TNFR2 humanized mouse model was used for evaluation in this example. The specific method was as follows: MC-38 cells (mouse colon cancer cells, Cobioer Biosciences, CBP60825) in the logarithmic growth phase were taken, and inoculated subcutaneously into each mouse at 1×10⁶ cells per mouse, with TNFR2 humanized mice (Biocytogen, 110032, female, 5-6 weeks old) selected; the mice were randomly grouped when the tumors grew to 100 mm³, with 6-8 mice in each group; the administration method was intraperitoneal injection, twice a week for 3 weeks.

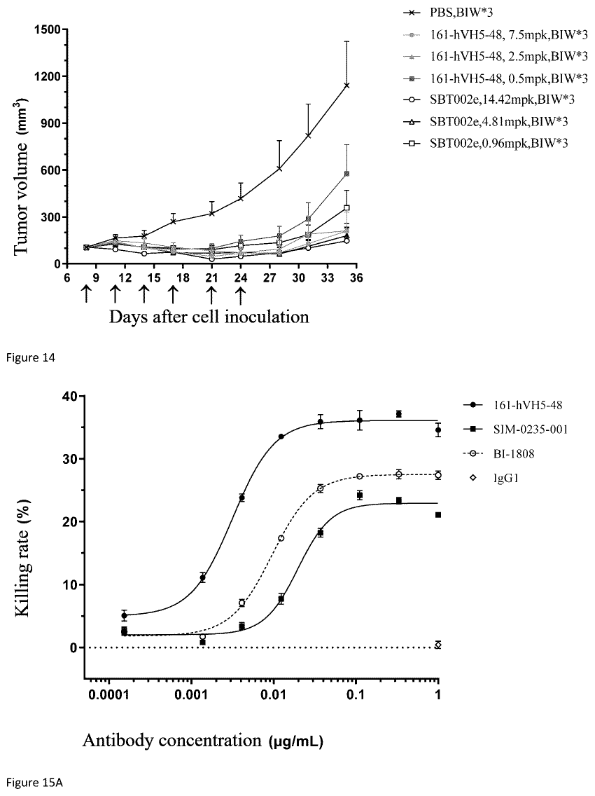

The results were as shown in FIG. 14 and Table 4, indicating that the tumor suppression effects of the affinity-matured molecule 161-hVH5-48 and the control antibody SBT002e were comparable at high and medium doses, but the number of the mice with complete regression of tumor in the antibody 161-hVH5-48 group was greater than that in the control antibody group, and especially at the medium dose, the number of the mice with complete regression of tumor in the antibody 161-hVH5-48 group was much greater than that in the control antibody group.

TABLE 4
Number of mice with complete regression of tumor

| | Number of mice with complete regression | | | | | |
|---|---|---|---|---|---|---|
| | (CR)/total number of mice | | | | | |
| Group | D 17 | D 21 | D 24 | D 28 | D 31 | D 35 |
| 161-hVH5-48-7.5 mpk | 1/6 | 2/6 | 2/5 | 2/4 | 2/4 | 2/4 |
| 161-hVH5-48-2.5 mpk | 0/6 | 0/6 | 0/6 | 3/6 | 3/6 | 3/6 |
| 161-hVH5-48-0.5 mpk | 0/6 | 1/6 | 1/6 | 1/6 | 1/6 | 1/6 |
| SBT002e-14.42 mpk | 0/6 | 0/6 | 1/6 | 1/6 | 1/6 | 1/6 |
| SBT002e-4.81 mpk | 0/6 | 0/6 | 0/6 | 1/6 | 1/6 | 1/6 |
| SBT002e-0.96 mpk | 0/6 | 0/6 | 1/6 | 1/6 | 1/6 | 1/6 |

### Examples 18. ADCC Effect of Affinity-Matured Molecule

To detect the ADCC effect of the affinity-matured molecule, an in vitro ADCC model was used for evaluation in this example. The specific method was as follows:
the density of the huTNFR2-HEK293 cell suspension was adjusted to 2×10⁵ cells/ml and the cell suspension was added to a 96-well round-bottom plate at 50 μL/well; gradiently diluted affinity-matured molecule 161-hVH5-48, control antibody SIM-0235-001 and control antibody BI-1808 were then added at 50 μL/well, and incubated at 37° C. for 20 min; the density of the PBMC (Allcells, NF0074) suspension resuscitated one day earlier was adjusted to 5×10⁵ cells/ml, and the suspension was further added to the preincubated 96-well round-bottom plate at 50 μL/well and incubated at 37° C. for 4 h; after the incubation was completed, an LDH detection reagent (Shanghai Beyotime Biotechnology Co., Ltd., C0017) was further added at 60 μL/well and placed at room temperature for 1 h; after the incubation was completed, the signal value at OD490 was read by a microplate reader and the killing rate was calculated.

The detection results were as shown in FIG. 15A, indicating that the affinity-matured molecule 161-hVH5-48 had an ED50 of 0.0032 μg/mL and a maximum killing rate of up to 36%; the positive antibody SIM-0235-001 (Simcere Pharmaceutical Co., Ltd., WO 2021023098A1) had an ED50 of 0.0196 μg/mL and a maximum killing rate of up to 23%; the positive antibody BI-1808 (BioInvent, WO 2020089474A1) had an ED50 of 0.0095 μg/mL and a maximum killing rate of up to 28%; the ADCC effect of the affinity-matured molecule 161-hVH5-48 was much better than that of the control antibodies.

On the basis of the same method, the ADCC effect of the antibody on the huTNFR2-Jurkat cells was also detected in this example. The detection results were as shown in FIG. 15B, indicating that the ADCC effect of the affinity-matured molecule 161-hVH5-48 was much better than that of the control antibodies.

### Example 19 Crystal Structure Analysis of Detailed Epitopes

To determine the epitopes of 161-hVH5-48 binding to TNFR2, a complex crystal produced by complexing the TNFR2 with 161-hVH5-48 was prepared and the binding epitopes were analyzed by X-ray diffraction in this example.

Specifically, for the expression of an antigen protein, a TNFR2 (33-205 aa) protein was expressed by prokaryotic E. coli, an inclusion body protein expressed by E. coli was purified using a dilution renaturation method, and then the verification was performed using a molecular sieve Superdex 75. For the expression of an antibody, the full-length expression of the 161-hVH5-48 nanobody was performed by a CHO eukaryotic expression system, the effect of Fc on protein crystallization was removed using a papain digestion method, and the antibody was purified by a molecular sieve at the same time. After the preparation of the antigen and the antibody was completed, the renatured antigen protein TNFR2 (33-205 aa) and the antibody 161-hVH5-48 in which the Fc was subjected to enzyme digestion were incubated at 4° C. overnight, and then the complex was prepared by a molecular sieve Superdex 75. Subsequently, the crystal growing under specific conditions was obtained through protein crystal screening. The quality of the crystal was improved in terms of precipitant, salt concentration, pH, protein concentration, etc. through crystal growth optimization at a later stage. Finally, the diffraction pattern of the protein crystal was obtained through X-ray crystallographic diffraction, and software such as HKL3000, CCP4, Coot, and Phenix were used for phase analysis and model building. On the basis of structural analysis of the antigen-antibody complex, the key amino acid sites were determined using software PDBePISA and Chimera.

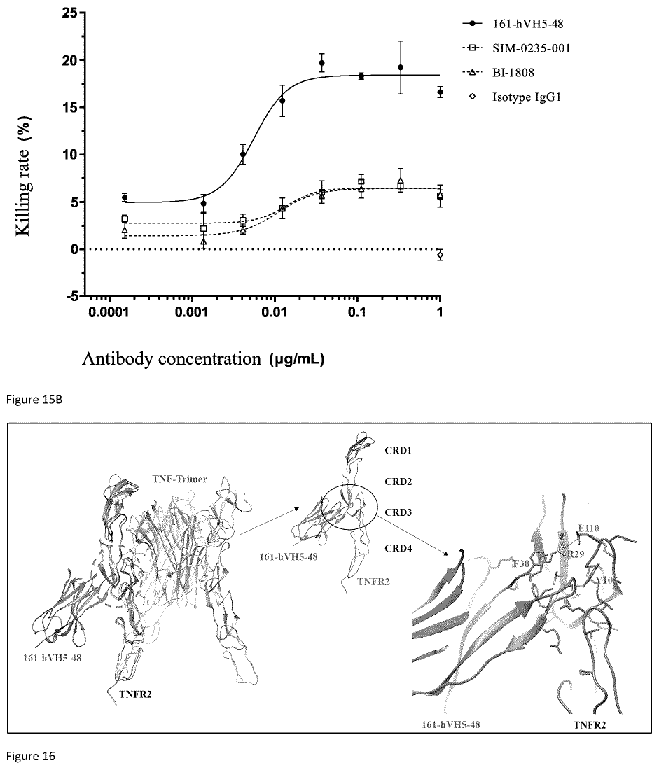

The results were as shown in FIG. 16, indicating that the 161-hVH5-48 antibody was predominantly bound in the groove of the CRD3 domain of the antigen TNFR2, and the antigenic epitopes critical for the binding included 13 amino acids such as V83, E84, T85, T97, C98, P100, G101, K108, E110, C112, G131, T132 and E133; the key antibody interaction amino acid sites included 14 amino acids such as R29, F30, N32, R53, E99, S101, Q102, L103, G104, Y105, A106, F107, R108 and D109; specific antigen-antibody interactions included salt bond (E110-R29), hydrogen bond (E84-R53, T97-G104, C98-Y105, C98-F107, K108-Y105, G131-R108, T132-R108, E133-S101, E133-Q102, E133-L103, E133-G104, E133-Y105 and E133-A106) and hydrophobic interactions. By comparison with the known TNF-TNFR2 crystal structure (PDB: 3ALQ), it was found that a TNF trimer was bound to its receptor TNFR2 mainly through CRD2 and CRD3, which had a certain overlap with the region CRD3 that was bound by the 161-hVH5-48 antibody. Therefore, it was believed that the binding to the 161-hVH5-48 antibody might hinder the TNF trimer from normally binding to the TNFR2 receptor, suggesting that the 161-hVH5-48 antibody might function as a non-classical blocking antibody.

### Exemplary Sequence

SEQ ID NO: 1 TNFα extracellular region
VRSSSRTPSDKPVAHVVANPQAEGQLQWLNRRANALLANGVELRDNQLVVPSEGLYLIYSQVLFKGQGCPST
HVLLTHTISRIAVSYQTKVNLLSAIKSPCQRETPEGAEAKPWYEPIYLGGVFQLEKGDRLSAEINRPDYLDFAE
SGQVYFGIIAL
SEQ ID NO: 2 hIgG1 Fc
EPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGNVFSCSVMHE
ALHNHYTQKSLSLSPGK
SEQ ID NO: 3 NB92-161 CDR1
GSIFSINDMG
SEQ ID NO: 4 NB92-161 CDR2
AIGRGGGSTN
SEQ ID NO: 5 NB92-161 CDR3
EISQLTWAFRDY
SEQ ID NO: 6 NB92-161 VHH
EVQLVESGGGLVQPGGSLRLSCAASGSIFSINDMGWYRQAPGKQRELVAAIGRGGGSTNYADSVKGRFTISR
DNAKNTVYLQMNSLKPEDTAVYYCHAEISQLTWAFRDYWGQGTQVTVSS
SEQ ID NO: 7 NB92-161-hVH5
EVQLVESGGGLVQPGGSLRLSCAASGSIFSINDMGWYRQAPGKGLELVAAIGRGGGSTNYADSVKGRFTISR
DNAKNTLYLQMNSLRAEDTAVYYCHAEISQLTWAFRDYWGQGTLVTVSS
SEQ ID NO: 8 NB92-161-hVH4
EVQLVESGGGLVQPGGSLRLSCAASGSIFSINDMGWYRQAPGKQRELVAAIGRGGGSTNYADSVKGRFTISR
DNAKNTLYLQMNSLRAEDTAVYYCHAEISQLTWAFRDYWGQGTLVTVSS
SEQ ID NO: 9 TNFR2 full-length protein
MAPVAVWAALAVGLELWAAAHALPAQVAFTPYAPEPGSTCRLREYYDQTAQMCCSKCSPGQHAKVFCTK
TSDTVCDSCEDSTYTQLWNWVPECLSCGSRCSSDQVETQACTREQNRICTCRPGWYCALSKQEGCRLCAPL
RKCRPGFGVARPGTETSDVVCKPCAPGTFSNTTSSTDICRPHQICNVVAIPGNASMDAVCTSTSPTRSMAPGA
VHLPQPVSTRSQHTQPTPEPSTAPSTSFLLPMGPSPPAEGSTGDFALPVGLIVGVTALGLLIIGVVNCVIMTQV
KKKPLCLQREAKVPHLPADKARGTQGPEQQHLLITAPSSSSSSLESSASALDRRAPTRNQPQAPGVEASGAGE
ARASTGSSDSSPGGHGTQVNVTCIVNVCSSSDHSSQCSSQASSTMGDTDSSPSESPKDEQVPFSKEECAFRSQL
ETPETLLGSTEEKPLPLGVPDAGMKPS
SEQ ID NO: 10 161-hVH5-1 CDR1
GSIFSINDMG
SEQ ID NO: 11 161-hVH5-1 CDR2
VHGRGGGSTN
SEQ ID NO: 12 161-hVH5-1 CDR3
EISQLTWAFRDY
SEQ ID NO: 13 161-hVH5-1 VHH
EVQLVESGGGLVQPGGSLRLSCAASGSIFSINDMGWYRQAPGKGLELVAVHGRGGGSTNYADSVKGRFTIS
RDNAKNTLYLQMNSLRAEDTAVYYCHAEISQLTWAFRDYWGQGTLVTVSS
SEQ ID NO: 14 161-hVH5-3 CDR1
GSIFSINDMG
SEQ ID NO: 15 161-hVH5-3 CDR2
AIGRGRRSTN
SEQ ID NO: 16 161-hVH5-3 CDR3
EISQLSFAFRDY
SEQ ID NO: 17 161-h VH5-3 VHH
EVQLVESGGGLVQPGGSLRLSCAASGSIFSINDMGWYRQAPGKGLELVAAIGRGRRSTNYADSVKGRFTISR
DNAKNTLYLQMNSLRAEDTAVYYCHAEISQLSFAFRDYWGQGTLVTVSS
SEQ ID NO: 18 161-hVH5-8 CDR1
GSIFSINDMG
SEQ ID NO: 19 161-hVH5-8 CDR2
AIGRGGQRTN
SEQ ID NO: 20 161-hVH5-8 CDR3
EISQLSFAFRDY
SEQ ID NO: 21 161-hVH5-8 VHH
EVQLVESGGGLVQPGGSLRLSCAASGSIFSINDMGWYRQAPGKGLELVAAIGRGGQRTNYADSVKGRFTISR
DNAKNTLYLQMNSLRAEDTAVYYCHAEISQLSFAFRDYWGQGTLVTVSS
SEQ ID NO: 22 161-hVH5-10 CDR1
GSIFSILRMG
SEQ ID NO: 23 161-hVH5-10 CDR2
AIGRTRGSTN
SEQ ID NO: 24 161-hVH5-10 CDR3
EISQLTWAFRDY
SEQ ID NO: 25 161-hVH5-10 VHH
EVQLVESGGGLVQPGGSLRLSCAASGSIFSILRMGWYRQAPGKGLELVAAIGRTRGSTNYADSVKGRFTISR
DNAKNTLYLQMNSLRAEDTAVYYCHAEISQLTWAFRDYWGQGTLVTVSS
SEQ ID NO: 26 161-hVH5-19 CDR1
GSIWSINDMG
SEQ ID NO: 27 161-hVH5-19 CDR2
AIGRGGGSTN
SEQ ID NO: 28 161-hVH5-19 CDR3
EISQLTWAFLDY
SEQ ID NO: 29 161-hVH5-19 VHH
EVQLVESGGGLVQPGGSLRLSCAASGSIWSINDMGWYRQAPGKGLELVAAIGRGGGSTNYADSVKGRFTIS
RDNAKNTLYLQMNSLRAEDTAVYYCHAEISQLTWAFLDYWGQGTLVTVSS
SEQ ID NO: 30 161-hVH5-22 CDR1
GSIFSINDMG
SEQ ID NO: 31 161-hVH5-22 CDR2
AIGRRPGSTN
SEQ ID NO: 32 161-hVH5-22 CDR3
EISQLSFAFRDY
SEQ ID NO: 33 161-hVH5-22 VHH
EVQLVESGGGLVQPGGSLRLSCAASGSIFSINDMGWYRQAPGKGLELVAAIGRRPGSTNYADSVKGRFTISR
DNAKNTLYLQMNSLRAEDTAVYYCHAEISQLSFAFRDYWGQGTLVTVSS
SEQ ID NO: 34 161-hVH5-24 CDR1
GSIFSILSMG
SEQ ID NO: 35 161-hVH5-24 CDR2
AIGRGGGSLQ
SEQ ID NO: 36 161-hVH5-24 CDR3
EISQLTWAFRDY
SEQ ID NO: 37 161-hVH5-24 VHH
EVQLVESGGGLVQPGGSLRLSCAASGSIFSILSMGWYRQAPGKGLELVAAIGRGGGSLQYADSVKGRFTISR
DNAKNTLYLQMNSLRAEDTAVYYCHAEISQLTWAFRDYWGQGTLVTVSS
SEQ ID NO: 38 161-hVH5-36 CDR1
GSIFSINDMG
SEQ ID NO: 39 161-hVH5-36 CDR2
AIGRGSVSTN
SEQ ID NO: 40 161-hVH5-36 CDR3
EISQLTYAFRDY
SEQ ID NO: 41 161-hVH5-36 VHH
EVQLVESGGGLVQPGGSLRLSCAASGSIFSINDMGWYRQAPGKGLELVAAIGRGSVSTNYADSVKGRFTISR
DNAKNTLYLQMNSLRAEDTAVYYCHAEISQLTYAFRDYWGQGTLVTVSS
SEQ ID NO: 42 161-hVH5-37 CDR1
GSIFSINDMG
SEQ ID NO: 43 161-hVH5-37 CDR2
AIGRGGFSTN
SEQ ID NO: 44 161-hVH5-37 CDR3
EISQLSFAFRDY
SEQ ID NO: 45 161-hVH5-37 VHH
EVQLVESGGGLVQPGGSLRLSCAASGSIFSINDMGWYRQAPGKGLELVAAIGRGGFSTNYADSVKGRFTISR
DNAKNTLYLQMNSLRAEDTAVYYCHAEISQLSFAFRDYWGQGTLVTVSS
SEQ ID NO: 46 161-hVH5-48 CDR1
GSIRFINDMG
SEQ ID NO: 47 161-hVH5-48 CDR2
AIGRGGGSTN
SEQ ID NO: 48 161-hVH5-48 CDR3
EISQLGYAFRDY
SEQ ID NO: 49 161-hVH5-48 VHH
EVQLVESGGGLVQPGGSLRLSCAASGSIRFINDMGWYRQAPGKGLELVAAIGRGGGSTNYADSVKGRFTISR
DNAKNTLYLQMNSLRAEDTAVYYCHAEISQLGYAFRDYWGQGTLVTVSS
SEQ ID NO: 50 161-hVH5-49 CDR1
GSIFSINDMG
SEQ ID NO: 51 161-hVH5-49 CDR2
ALARGGGSTN
SEQ ID NO: 52 161-hVH5-49 CDR3
EISQLSFAFRDY
SEQ ID NO: 53 161-hVH5-49 VHH
EVQLVESGGGLVQPGGSLRLSCAASGSIFSINDMGWYRQAPGKGLELVAALARGGGSTNYADSVKGRFTISR
DNAKNTLYLQMNSLRAEDTAVYYCHAEISQLSFAFRDYWGQGTLVTVSS
SEQ ID NO: 54 CDR1
G-S-I-Xaa1-Xaa2-I-Xaa3-Xaa4-M-G

wherein Xaa1 is F, W or R, Xaa2 is S or F, Xaa3 is N or L, and Xaa4 is S, D or R.

CDR2
SEQ ID NO: 55
Xaa5-Xaa6-Xaa7-R-Xaa8-Xaa9-Xaa10-Xaa11-Xaa12-
Xaa13

wherein Xaa5 is A or V, Xaa6 is I, L or H, Xaa7 is G or A, Xaa8 is G, R or T, Xaa9 is G, R, P or S, Xaa10 is G, Q, R, F or V, Xaa11 is S or R, Xaa12 is T or L, and Xaa13 is N or Q.

CDR3
SEQ ID NO: 56
E-I-S-Q-L-Xaa14-Xaa15-A-F-Xaa16-D-Y

wherein Xaa14 is T, S or G, Xaa15 is W, F or Y, and Xaa16 is R or L.

1. A TNFR2 binding molecule, comprising at least one single domain antibody (sdAb) moiety that specifically binds to TNFR2, wherein the sdAb moiety comprises, from the N-terminus to the C-terminus, three complementarity determining regions, respectively CDR1, CDR2 and CDR3, wherein:
(a) the CDR1 comprises the amino acid sequence of SEQ ID NO: 3, or a variant with 1 or 2 amino acid changes in the amino acid sequence of SEQ ID NO: 3,
(b) the CDR2 comprises the amino acid sequence of SEQ ID NO: 4, or a variant with 1 or 2 amino acid changes in the amino acid sequence of SEQ ID NO: 4, and
(c) the CDR3 comprises the amino acid sequence of SEQ ID NO: 5, or a variant with 1 or 2 amino acid changes in the amino acid sequence of SEQ ID NO: 5,
wherein the amino acid change is an addition, deletion or substitution of an amino acid, and the binding molecule comprising the above-mentioned change at least retains the binding ability to TNFR2.

2. The TNFR2 binding molecule according to claim 1, wherein the sdAb moiety comprises:
(a) CDR1 comprising the amino acid sequence of SEQ ID NO: 54:
(SEQ ID NO: 54)
G-S-I-Xaa1-Xaa2-I-Xaa3-Xaa4-M-G
wherein
Xaa1 is F, W or R, Xaa2 is S or F, Xaa3 is N or L, and Xaa4 is S, D or R;
(b) CDR2 comprising the amino acid sequence of SEQ ID NO: 55:
(SEQ ID NO: 55)
Xaa5-Xaa6-Xaa7-R-Xaa8-Xaa9-Xaa10-Xaa11-Xaa12-
Xaa13
wherein
Xaa5 is A or V, Xaa6 is I, L or H, Xaa7 is G or A, Xaa8 is G, R or T, Xaa9 is G, R, P or S, Xaa10 is G, Q, R, For V, Xaa11 is S or R, Xaa12 is T or L, and Xaa13 is N or Q; and
(c) CDR3 comprising the amino acid sequence of SEQ ID NO: 56:
(SEQ ID NO: 56)
E-I-S-Q-L-Xaa14-Xaa15-A-F-Xaa16-D-Y
wherein
Xaa14 is T, S or G, Xaa15 is W, F or Y, and Xaa16 is R or L.

3. The TNFR2 binding molecule according to claim 2, wherein the sdAb moiety comprises CDR1, CDR2 and CDR3 selected from any one of the following groups:
(a) CDR1 comprising the amino acid sequence of SEQ ID NO: 3; CDR2 comprising the amino acid sequence of SEQ ID NO: 4; and CDR3 comprising the amino acid sequence of SEQ ID NO: 5;
(b) CDR1 comprising the amino acid sequence of SEQ ID NO: 10; CDR2 comprising the amino acid sequence of SEQ ID NO: 11; and CDR3 comprising the amino acid sequence of SEQ ID NO: 12;
(c) CDR1 comprising the amino acid sequence of SEQ ID NO: 14; CDR2 comprising the amino acid sequence of SEQ ID NO: 15; and CDR3 comprising the amino acid sequence of SEQ ID NO: 16;
(d) CDR1 comprising the amino acid sequence of SEQ ID NO: 18; CDR2 comprising the amino acid sequence of SEQ ID NO: 19; and CDR3 comprising the amino acid sequence of SEQ ID NO: 20;
(e) CDR1 comprising the amino acid sequence of SEQ ID NO: 22; CDR2 comprising the amino acid sequence of SEQ ID NO: 23; and CDR3 comprising the amino acid sequence of SEQ ID NO: 24;
(f) CDR1 comprising the amino acid sequence of SEQ ID NO: 26; CDR2 comprising the amino acid sequence of SEQ ID NO: 27; and CDR3 comprising the amino acid sequence of SEQ ID NO: 28;
(g) CDR1 comprising the amino acid sequence of SEQ ID NO: 30; CDR2 comprising the amino acid sequence of SEQ ID NO: 31; and CDR3 comprising the amino acid sequence of SEQ ID NO: 32;
(h) CDR1 comprising the amino acid sequence of SEQ ID NO: 34; CDR2 comprising the amino acid sequence of SEQ ID NO: 35; and CDR3 comprising the amino acid sequence of SEQ ID NO: 36;
(i) CDR1 comprising the amino acid sequence of SEQ ID NO: 38; CDR2 comprising the amino acid sequence of SEQ ID NO: 39; and CDR3 comprising the amino acid sequence of SEQ ID NO: 40;
(j) CDR1 comprising the amino acid sequence of SEQ ID NO: 42; CDR2 comprising the amino acid sequence of SEQ ID NO: 43; and CDR3 comprising the amino acid sequence of SEQ ID NO: 44;
(k) CDR1 comprising the amino acid sequence of SEQ ID NO: 46; CDR2 comprising the amino acid sequence of SEQ ID NO: 47; and CDR3 comprising the amino acid sequence of SEQ ID NO: 48;
(l) CDR1 comprising the amino acid sequence of SEQ ID NO: 50; CDR2 comprising the amino acid sequence of SEQ ID NO: 51; and CDR3 comprising the amino acid sequence of SEQ ID NO: 52.

4. The TNFR2 binding molecule according to claim 1, wherein the sdAb moiety comprises
(i) an amino acid sequence selected from any one of amino acid sequences of SEQ ID NOs: 6, 7, 8, 13, 17, 21, 25, 29, 33, 37, 41, 45, 49 and 53; or
(ii) an amino acid sequence having at least 85%, 90%, 91%, 92%, 93%, 94%, 95%, 96%, 97%, 98% or 99% identity to the amino acid sequence selected from any one of amino acid sequences of SEQ ID NOs: 6, 7, 8, 13, 17, 21, 25, 29, 33, 37, 41, 45, 49 and 53;
preferably, the sdAb moiety is camelid VHH, partially humanized or fully humanized VHH, or chimeric VHH.

5. The TNFR2 binding molecule according to claim 1, wherein the sdAb moiety is linked to another protein domain at the N-terminus or C-terminus, for example, to the Fc region of an immunoglobulin, for example, to the Fc region from IgG, such as IgG1, IgG2, IgG3 or IgG 4; or, for example, the sdAb moiety is linked to a fluorescent protein.

6. The TNFR2 binding molecule according to claim 1, having one or more of the following properties:
(1) binding to human TNFR2 with high affinity, for example, the EC50 for the binding between the TNFR2 binding molecule and cell surface TNFR2 is about 0.01 μg/mL to about 1 μg/mL, such as about 0.1 μg/mL to about 0.6 μg/mL;
(2) not substantially blocking the binding of TNFα to TNFR2;
(3) inhibiting the TNFR2 signaling pathway, for example, signaling mediated by TNFR2 is inhibited in TNFR2-expressing cells such as Treg cells (e.g., Treg cells with high expression of CD25), myeloid-derived suppressing cells (MDSCs) and/or TNFR2+ cancer cells;
(4) not substantially affecting the proliferation of Treg cells in PBMCs;
(5) inhibiting tumor growth in vivo.

7. The TNFR2 binding molecule according to claim 1, wherein the TNFR2 binding molecule is a bispecific or multispecific antibody, preferably, the bispecific antibody molecule specifically binds to a TNFR2 molecule and a second target protein, and the second target protein is selected from, for example, a tumor antigen (such as a tumor associated antigen and a tumor specific antigen), an immunoregulatory receptor, and an immune checkpoint molecule, such as CTLA-4, TIM-3 or LAG-3.

8. The TNFR2 binding molecule according to claim 1, wherein the TNFR2 binding molecule binds in the groove of the CRD3 domain of TNFR2, for example, the TNFR2 binding molecule binds to an epitope of TNFR2 that comprises amino acid residues at positions 83, 84, 85, 97, 98, 100, 101, 108, 110, 112, 131, 132 and 133, for example, the TNFR2 binding molecule binds to an epitope of TNFR2 as shown in SEQ ID NO: 9 that comprises amino acid residues at positions V83, E84, T85, T97, C98, P100, G101, K108, E110, C112, G131, T132 and E133.

9. An isolated nucleic acid encoding the TNFR2 binding molecule according to claim 1.

10. A vector comprising the nucleic acid according to claim 9, wherein preferably the vector is an expression vector, for example, a pcDNA3.4-TOPO vector.

11. A host cell comprising the nucleic acid according to claim 9, wherein preferably the host cell is a prokaryotic cell or a eukaryotic cell, more preferably the host cell is selected from an E. coli cell, a yeast cell or a mammalian cell, and most preferably the host cell is an HEK293 cell or a CHO cell.

12. A method for preparing the TNFR2 binding molecule according to claim 1, wherein the method comprises culturing a host cell under conditions suitable for expressing a nucleic acid in the host cell encoding the TNFR2 binding molecule, and optionally isolating the TNFR2 binding molecule, and optionally the method further comprises recovering the TNFR2 binding molecule from the host cell.

13. A pharmaceutical composition, comprising the TNFR2 binding molecule according to claim 1, and optionally a pharmaceutical auxiliary material.

14. A combined product, comprising the TNFR2 binding molecule according to claim 1, and other therapeutic agents, and/or optionally a pharmaceutical auxiliary material, wherein preferably, the other therapeutic agents are selected from chemotherapeutic agents and other antibodies (such as anti-PD-1 antibodies or anti-PD-L1 antibodies).

15. A method for treating a disease associated with TNFR2 in a subject, comprising administering to the subject a therapeutically effective amount of the TNFR2 binding molecule according to claim 1, wherein the disease associated with TNFR2 is, for example, a cancer.

16. A kit for detecting TNFR2 in a sample, wherein the kit comprises the TNFR2 binding molecule according to claim 1, and is used for performing the following steps:
(a) contacting the sample with the TNFR2 binding molecule; and
(b) detecting the formation of a complex of the TNFR2 binding molecule and TNFR2; optionally, the TNFR2 binding molecule is detectably labeled.

17. An epitope peptide of TNFR2, wherein the epitope peptide is located in the groove of the CRD3 domain of TNFR2, for example, the epitope peptide is an epitope peptide of TNFR2 that comprises amino acid residues at positions 83, 84, 85, 97, 98, 100, 101, 108, 110, 112, 131, 132 and 133, for example, the epitope peptide is an epitope peptide of TNFR2 as shown in SEQ ID NO: 9 that comprises amino acid residues at positions V83, E84, T85, T97, C98, P100, G101, K108, E110, C112, G131, T132 and E133.

18. The epitope peptide of TNFR2 according to claim 17, wherein the amino acid residue sites at which a TNFR2 binding molecule interacts with the epitope peptide include amino acid residues at positions 29, 30, 32, 53, 99, 101, 102, 103, 104, 105, 106, 107, 108 and 109, for example, the amino acid residue sites at which the TNFR2 binding molecule interacts with the epitope peptide include amino acid residues at positions R29, F30, N32, R53, E99, S101, Q102, L103, G104, Y105, A106, F107, R108 and D109 in a sequence as shown in SEQ ID NO: 49.

19. A TNFR2 binding molecule, wherein the TNFR2 binding molecule binds in the groove of the CRD3 domain of TNFR2, for example, the TNFR2 binding molecule binds to an epitope of TNFR2 that comprises amino acid residues at positions 83, 84, 85, 97, 98, 100, 101, 108, 110, 112, 131, 132 and 133, for example, the TNFR2 binding molecule binds to an epitope of TNFR2 as shown in SEQ ID NO: 9 that comprises amino acid residues at positions V83, E84, T85, T97, C98, P100, G101, K108, E110, C112, G131, T132 and E133.
